(19) United States
(12) Patent Application Publication
(10) Pub. No.: US 2025/0057826 A1
(43) Pub. Date: Feb. 20, 2025

(54) WEEKLY REGIMEN FOR HIV

(71) Applicant: Gilead Sciences, Inc., Foster City, CA (US)

(72) Inventors: Jared Baeten, San Francisco, CA (US); Christoph C. Carter, Oakland, CA (US); Moupali Das, San Francisco, CA (US); Rameshraja Palaparthy, Foster City, CA (US); Martin S. Rhee, Foster City, CA (US); Abdul Naveed Shaik, Union City, CA (US); Renu Singh, Silver Spring, MD (US)

(21) Appl. No.: 18/785,641

(22) Filed: Jul. 26, 2024

Related U.S. Application Data
(60) Provisional application No. 63/529,468, filed on Jul. 28, 2023.

(51) Int. Cl. A61K 31/4439; A61K 9/00; A61K 9/20; A61K 9/28; A61P 31/18

(52) U.S. Cl. CPC A61K 31/4439; A61K 9/0053; A61K 9/2018; A61K 9/2027; A61K 9/2031; A61K 9/2054; A61K 9/2853; A61P 31/18

(57) ABSTRACT

The present disclosure provides methods of treating or preventing human immunodeficiency virus (HIV) infection in a patient, comprising orally administering to the patient a therapeutically effective amount of an HIV capsid inhibitor, or a pharmaceutically acceptable salt thereof.

### CROSS-REFERENCE TO RELATED APPLICATIONS

This application claims the benefit of U.S. Provisional Application No. 63/529,468, filed on Jul. 28, 2023, the entire content of which is hereby incorporated by reference in its entirety.

### TECHNICAL FIELD

The present disclosure provides methods of treating or preventing human immunodeficiency virus (HIV) infection in a patient, comprising orally administering to the patient a therapeutically effective amount of an HIV capsid inhibitor, or a pharmaceutically acceptable salt thereof.

### BACKGROUND

The viral capsid protein (CA) is essential for multiple stages of the HIV life cycle. During viral maturation following the processing of Gag polyprotein by the HIV protease, CA self-assembles into the conical shaped core characteristic of mature HIV-1 virions. Contained within this capsid core are the viral RNA, nucleocapsid, reverse transcriptase, and integrase. Failure to generate a suitable core precludes infectivity. In addition, CA contributes to multiple essential processes during the early stages of HIV replication, including important roles in regulating proper capsid core disassembly (uncoating) kinetics to ensure efficient and productive viral DNA synthesis via coupled reverse transcription, and contributes to the active transport of pre-integration complexes into the nuclear compartment to support viral DNA integration into transcriptionally active loci. Defects in the proper function of capsid ultimately inhibit efficient nuclear uptake and integration of viral DNA into the host genome.

Human immunodeficiency virus type 1 infection is a life-threatening and serious disease of major public health significance, with approximately 38 million people infected worldwide and approximately 26 million on antiretroviral (ARV) treatment (UNAIDS. Global HIV & AIDS statistics, 2020 fact sheet). Advances in combination ARV therapy for HIV have led to significant improvements in morbidity and mortality by suppressing viral replication, preserving immunologic function, and averting disease progression to AIDS.

### SUMMARY

The present disclosure provides methods of treating or preventing human immunodeficiency virus (HIV) infection in a patient, comprising administering to the patient a compound of Formula Ia:

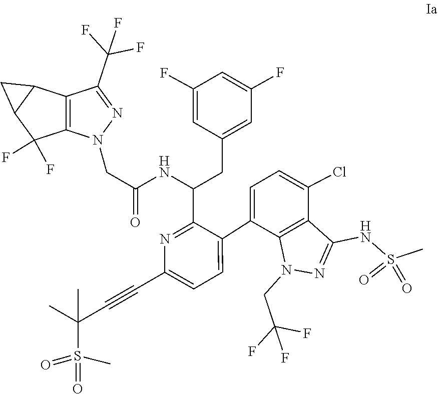 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the methods comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time, and
wherein the compound of Formula Ia, or a pharmaceutically acceptable salt thereof, is administered as a monotherapy.

The present disclosure further provides a method of treating or preventing human immunodeficiency virus (HIV) infection in a patient, comprising administering to the patient a compound of Formula Ia:

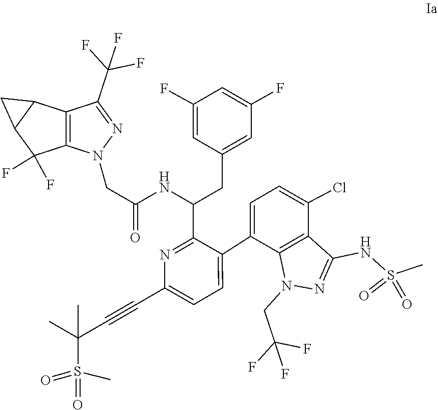 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein if the patient misses a maintenance dosage, the method further comprises orally administering to the patient about 200 mg to about 700 mg of the compound of Formula Ia, or a pharmaceutically acceptable salt thereof, for a third period of time, and resuming administration of the one or more maintenance dosages within about 1 day to about 7 days after the oral administration during the third period of time.

The present disclosure further provides a method of preventing human immunodeficiency virus (HIV) infection in a patient, comprising administering to the patient a compound of Formula Ia:

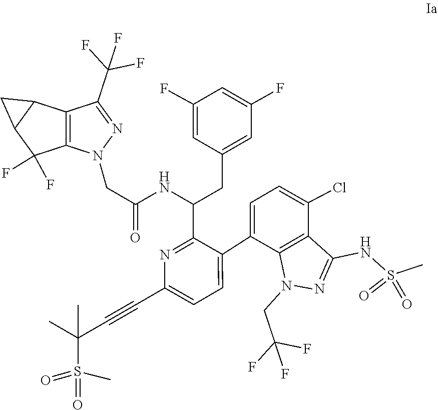 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein the method comprises pre-exposure prophylaxis (PrEP).

The present disclosure further provides a method of preventing human immunodeficiency virus (HIV) infection in a patient, comprising administering to the patient a compound of Formula Ia:

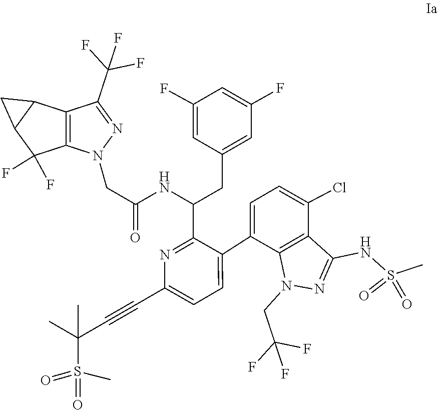 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein if the patient misses a maintenance dosage, the method further comprises orally administering to the patient about 200 mg to about 700 mg of the compound of Formula Ia, or a pharmaceutically acceptable salt thereof, for a third period of time, and resuming administration of the one or more maintenance dosages within about 1 day to about 7 days after the oral administration during the third period of time; and
wherein the method comprises pre-exposure prophylaxis (PrEP).

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DESCRIPTION OF DRAWINGS

FIG. 1 shows a summary of the lenacapavir structural pharmacokinetic (PK) model.

FIG. 2 shows modeled results of a lenacapavir oral dosing regimen.

FIG. 3 shows modeled results of a lenacapavir oral dosing regimen with one missed dose of the lenacapavir.

FIG. 4 shows modeled results of a lenacapavir oral dosing regimen with two missed doses of the lenacapavir.

### DETAILED DESCRIPTION

Lenacapavir, a human immunodeficiency virus type 1 (HIV-1) capsid inhibitor, is indicated for the treatment of HIV-1 infection (e.g., in heavily treatment-experienced adults with multidrug resistant HIV-1 infection failing their current antiretroviral regimen due to resistance, intolerance, or safety considerations). The recommended dosage of lenacapavir comprises an initiation dosage followed by maintenance dosing. The present application provides alternative oral dosing regimens of lenacapavir, which may be particularly useful, for example, in a pre-exposure prophylaxis (PrEP) dosing schedule.

In particular, the present disclosure relates to a method of treating or preventing human immunodeficiency virus (HIV) infection (e.g., HIV-1 and/or HIV-2) in a patient, comprising administering to the patient a compound of Formula Ia:

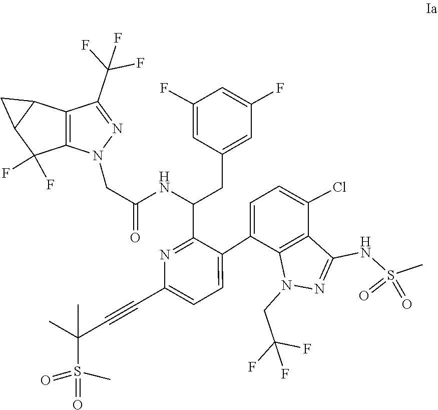 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time.

In some embodiments, the present disclosure relates to a method of treating or preventing human immunodeficiency virus (HIV) infection (e.g., HIV-1 and/or HIV-2) in a patient, comprising administering to the patient a compound of Formula Ia:

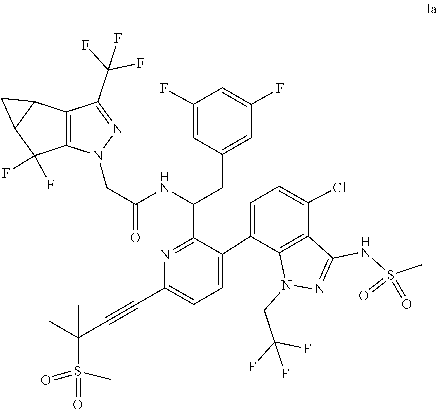 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time, and
wherein the compound of Formula Ia, or a pharmaceutically acceptable salt thereof, is administered as a monotherapy.

In some embodiments, the present disclosure relates to a method of treating or preventing human immunodeficiency virus (HIV) infection in a patient, comprising administering to the patient a compound of Formula Ia:

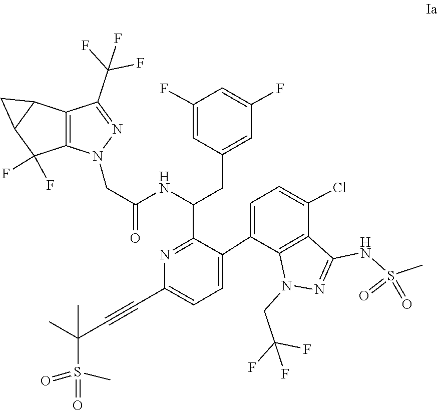 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein if the patient misses a maintenance dosage, the method further comprises orally administering to the patient about 200 mg to about 700 mg of the compound of Formula Ia, or a pharmaceutically acceptable salt thereof, for a third period of time, and resuming administration of the one or more maintenance dosages within about 1 day to about 7 days after the oral administration during the third period of time.

In some embodiments, the present disclosure relates to a method of preventing human immunodeficiency virus (HIV) infection in a patient, comprising administering to the patient a compound of Formula Ia:

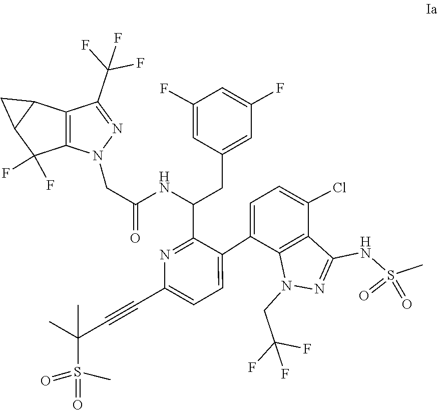 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time. In some embodiments of the previous embodiment, the method comprises pre-exposure prophylaxis (PrEP).

In some embodiments, the present disclosure relates to a method of preventing human immunodeficiency virus (HIV) infection in a patient, comprising administering to the patient a compound of Formula Ia:

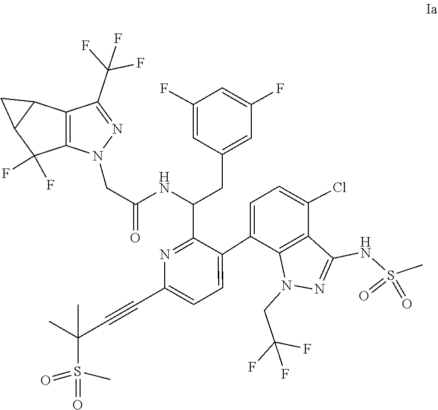 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein if the patient misses a maintenance dosage, the method further comprises orally administering to the patient about 200 mg to about 700 mg of the compound of Formula Ia, or a pharmaceutically acceptable salt thereof, for a third period of time, and resuming administration of the one or more maintenance dosages within about 1 day to about 7 days after the oral administration during the third period of time. In some embodiments of the previous embodiment, the method comprises pre-exposure prophylaxis (PrEP).

In any of the embodiments provided herein, the compound of Formula Ia is a compound of Formula Ib:

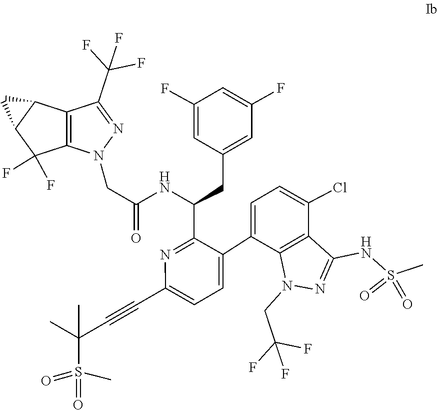 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof. The compound of Formula Ib may also be referred to as lenacapavir (or “LEN”) or N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide.

In some embodiments, the compound of Formula Ia or Ib, or a pharmaceutically acceptable salt thereof, is administered as a monotherapy (i.e., in the absence of an additional therapeutic agent). In some embodiments, the compound of Formula Ia or Ib, or a pharmaceutically acceptable salt thereof, is administered in combination with one or more additional therapeutic agents, such as anti-HIV agents.

In some embodiments, the method comprises administering the compound of Formula Ia or Ib, or a pharmaceutically acceptable salt thereof. In some embodiments, the method comprises administering the compound of Formula Ia, or a pharmaceutically acceptable salt thereof. In some embodiments, the method comprises administering the compound of Formula Ib, or a pharmaceutically acceptable salt thereof.

Synthesis and characterization of the compounds of Formula Ia and Formula Ib, and salts thereof, are described, for example, in US 20180051005 and US 20190300505, the contents of which are hereby incorporated by reference in their entireties. Various forms and/or uses of the compounds of Formula Ia and Ib are disclosed, for example, US 20190083478, US 20190084963, US 20200038389A1, and US 20210188815, the contents of which are hereby incorporated by reference in their entireties.

In some embodiments, the compound of Formula Ia or Ib is orally administered as the sodium salt. In some embodiments, the compound of Formula Ia is orally administered as the sodium salt. In some embodiments, the compound of Formula Ib is orally administered as the sodium salt.

In the absence of a specific reference to a particular pharmaceutically acceptable salt and/or solvate of the compound of Formula Ia or Ib, any dosages, whether expressed in milligrams or as % by weight, should be understood as referring to the amount of the free acid, i.e., the compound of Formula Ia or Formula Ib. For example, a reference to “50 mg” of Formula Ia or Ib, or a pharmaceutically acceptable salt thereof, refers to an amount of the compound of Formula Ia or Ib, or a pharmaceutically acceptable salt thereof, which provides the same amount of the compound of Formula Ia or Ib as 50 mg of the compound of Formula Ia or Ib free acid. In some embodiments, a dosage referring to 50 mg of Formula Ia or Ib contains about 51.1 mg of Formula Ia sodium salt.

In some embodiments, the compound provided herein (i.e., the compound of Formula Ia or Ib), or a pharmaceutically acceptable salt thereof, is administered orally in the form of one or more tablets as described herein.

In some embodiments, the compound of Formula Ia or Ib is administered once-daily (QD). In some embodiments, the compound of Formula Ia or Ib is orally administered once-daily (QD).

In some embodiments, the compound of Formula Ia or Ib is administered once-weekly (QW). In some embodiments, the compound of Formula Ia or Ib is orally administered once-weekly (QW).

In some embodiments, the compound of Formula Ia or Ib, or a pharmaceutically acceptable salt thereof, is administered in a dosage of from about 10 mg to about 2000 mg. In some embodiments, the compound of Formula Ia or Ib, or a pharmaceutically acceptable salt thereof, is administered in a dosage of from about 10 mg to about 3000 mg. In some embodiments, the compound of Formula Ia or Ib, or a pharmaceutically acceptable salt thereof, is administered in a dosage of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, the compound of Formula Ia or Ib, or a pharmaceutically acceptable salt thereof, is administered in a dosage of about 600 mg.

In some embodiments, the initiation dosage provided herein comprises orally administering to the patient about 600 mg of the compound of Formula Ia or Ib, or a pharmaceutically acceptable salt thereof, for the first period of time.

In some embodiments, the compound of Formula Ia or Ib, or a pharmaceutically acceptable salt thereof, is administered in a dosage of about 300 mg.

In some embodiments, the initiation dosage provided herein is administered as one or more tablets, each containing about 300 mg of the compound of Formula Ia or Ib, or a pharmaceutically acceptable salt thereof, for the first period of time.

In some embodiments, the first period of time is about one day to about two days. In some embodiments, the first period of time is one day. In some embodiments, the first period of time is two days.

In some embodiments, the oral administrations during the first period of time (i.e. the initiation dosage) are administered as one or two tablets (e.g., one or two tablets on the first day; one or two tablets on the second day; one or two tablets on the first day and one or two tablets on the second day; and the like). In some embodiments, the oral administrations during the first period of time (i.e., the initiation dosage) are each administered as two tablets. In some embodiments, the oral administrations during the initiation dosage are each administered as one tablet.

In some embodiments, the one or more maintenance dosages provided herein each comprise orally administering to the patient about 300 mg of the compound of Formula Ia or Ib, or a pharmaceutically acceptable salt thereof, for the second period of time.

In some embodiments, the one or more maintenance dosages provided herein are each administered as one tablet comprising about 300 mg of the compound of Formula Ia or Ib, or a pharmaceutically acceptable salt thereof, for the second period of time.

In some embodiments, the second period of time begins about six days to about eight days after the first oral administration of the initiation dosage.

In some embodiments, the second period of time begins about seven days after the first oral administration of the initiation dosage.

In some embodiments, the second period of time is at least about 1 week, about 2 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, or longer.

In some embodiments, the second period of time is at least about 1 year, 2 years, 3 years, 4 years, 5 years, 10 years, or longer.

In some embodiments, the second period of time is from about 1 week to about 25 years, for example, about 1 week to about 10 years, about 1 week to about 5 years, about 1 week to about 4 years, about 1 week to about 3 years, about 1 week to about 2 years, about 1 week to about 1 year, about 1 week to about 48 weeks, about 1 week to about 44 weeks, about 1 week to about 40 weeks, about 1 week to about 36 weeks, about 1 week to about 32 weeks, about 1 week to about 28 weeks, about 1 week to about 24 weeks, about 1 week to about 20 weeks, about 1 week to about 16 weeks, about 1 week to about 8 weeks, about 1 week to about 4 weeks, and the like.

In some embodiments, the second period of time comprises continuous administration of one or more maintenance dosages during the life of the patient.

In some embodiments, the third period of time begins within about one day to about fourteen days after the patient first misses a maintenance dosage, for example, within about one day, within about two days, within about three days, within about four days, within about five days, within about six days, within about seven days, within about eight days, within about nine days, within about ten days, within about eleven days, within about twelve days, within about thirteen days, or within about fourteen days.

In some embodiments, if the patient misses a maintenance dosage, the method further comprises orally administering to the patient about 300 mg to about 600 mg of the compound of Formula Ia or Ib, or a pharmaceutically acceptable salt thereof, for a third period of time, and resuming administration of the one or more maintenance dosages within about 1 day to about 7 days after the oral administration during the third period of time.

In some embodiments, if the patient misses a maintenance dosage, the method further comprises orally administering to the patient about 300 mg or about 600 mg of the compound of Formula Ia or Ib, or a pharmaceutically acceptable salt thereof, for a third period of time, and resuming administration of the one or more maintenance dosages within about 1 day to about 7 days after the oral administration during the third period of time.

In some embodiments, if the patient misses one or two maintenance dosages, the method further comprises orally administering to the patient about 300 mg or about 600 mg of the compound of Formula Ia or Ib, or a pharmaceutically acceptable salt thereof, for a third period of time, and resuming administration of the one or more maintenance dosages within about 1 day to about 7 days after the oral administration during the third period of time.

In some embodiments, if the patient misses one maintenance dosage, the method further comprises orally administering to the patient about 300 mg of the compound of Formula Ia or Ib, or a pharmaceutically acceptable salt thereof, for a third period of time, and resuming administration of the one or more maintenance dosages within about 1 day to about 7 days after the oral administration during the third period of time.

In some embodiments, if the patient misses two consecutive maintenance dosages, the method further comprises orally administering to the patient about 600 mg of the compound of Formula Ia or Ib, or a pharmaceutically acceptable salt thereof, for a third period of time, and resuming administration of the one or more maintenance dosages within about 1 day to about 7 days after the oral administration during the third period of time.

In some embodiments, the third period of time is about one day to about two days. In some embodiments, the third period of time is one day. In some embodiments, the third period of time is two days.

In some embodiments, the present disclosure provides a method of treating or preventing HIV in a patient, comprising administering to the patient a compound of Formula Ia:

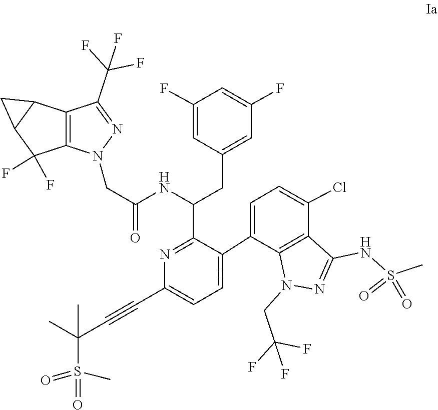 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week.

In some embodiments, the present disclosure provides a method of treating or preventing HIV in a patient, comprising administering to the patient a compound of Formula Ia:

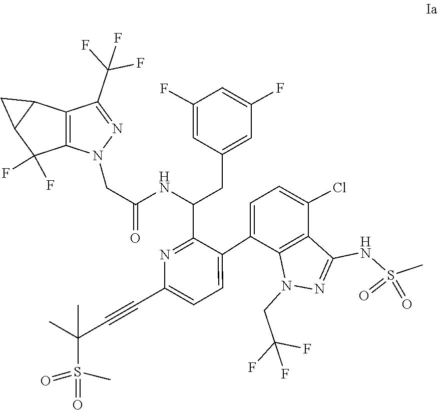 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week, and
wherein the compound of Formula Ia, or a pharmaceutically acceptable salt thereof, is administered as a monotherapy.

In some embodiments, the present disclosure provides a method of treating or preventing HIV in a patient, comprising administering to the patient a compound of Formula Ia:

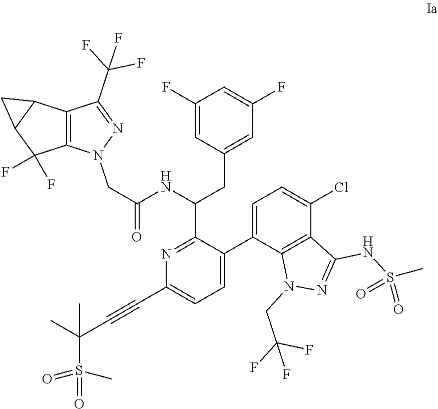 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses a maintenance dosage, the method further comprises orally administering to the patient about 300 mg of the compound of Formula Ia, or a pharmaceutically acceptable salt thereof, within a third period of time which is about one day to about fourteen days after the patient first misses the maintenance dosage, and resuming administration of the one or more maintenance dosages within about 1 day to about 7 days after the oral administration during the third period of time.

In some embodiments, the present disclosure provides a method of treating or preventing HIV in a patient, comprising administering to the patient a compound of Formula Ia:

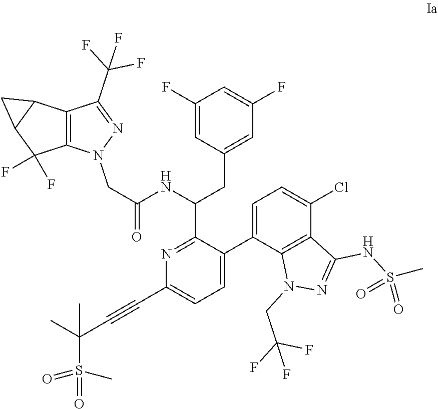 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses two consecutive maintenance dosages, the method further comprises orally administering to the patient about 600 mg of the compound of Formula Ia, or a pharmaceutically acceptable salt thereof, within a third period of time which is about one day to about fourteen days after the patient first misses the second maintenance dosage, and resuming administration of the one or more maintenance dosages within about 1 day to about 7 days after the oral administrations during the third period of time.

In some embodiments, the present disclosure provides a method of preventing HIV in a patient, comprising administering to the patient a compound of Formula Ia:

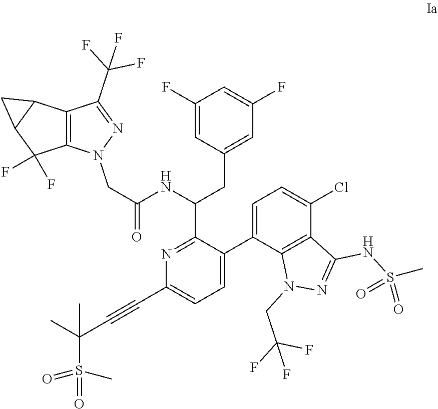 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein the method comprises pre-exposure prophylaxis (PrEP).

In some embodiments, the present disclosure provides a method of preventing HIV in a patient, comprising administering to the patient a compound of Formula Ia:

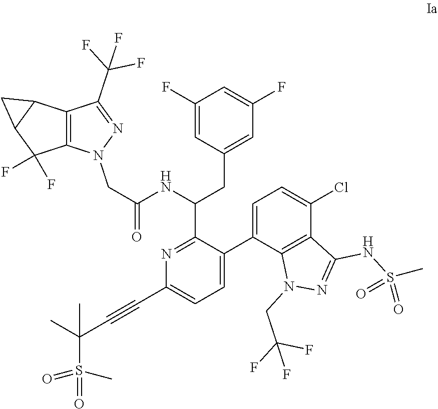 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses a maintenance dosage, the method further comprises orally administering to the patient about 300 mg of the compound of Formula Ia, or a pharmaceutically acceptable salt thereof, within a third period of time which is about one day to about fourteen days after the patient first misses the maintenance dosage, and resuming administration of the one or more maintenance dosages within about 1 day to about 7 days after the oral administration during the third period of time; and
wherein the method comprises pre-exposure prophylaxis (PrEP).

In some embodiments, the present disclosure provides a method of preventing HIV in a patient, comprising administering to the patient a compound of Formula Ia:

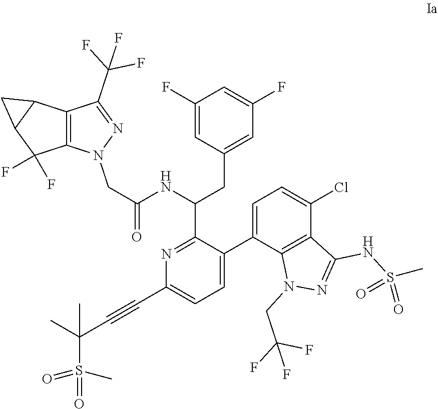 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt thereof, the method comprising:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses two consecutive maintenance dosages, the method further comprises orally administering to the patient about 600 mg of the compound of Formula Ia, or a pharmaceutically acceptable salt thereof, within a third period of time which is about one day to about fourteen days after the patient first misses the second maintenance dosage, and resuming administration of the one or more maintenance dosages within about 1 day to about 7 days after the oral administrations during the third period of time; and
wherein the method comprises pre-exposure prophylaxis (PrEP).

In some embodiments, the present disclosure provides a method of treating or preventing HIV in a patient, comprising administering to the patient a compound of Formula Ib:

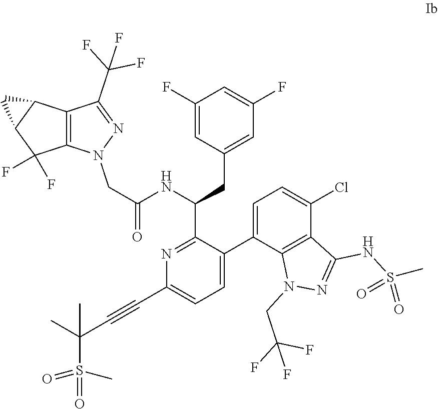 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week.

In some embodiments, the present disclosure provides a method of treating or preventing HIV in a patient, comprising administering to the patient a compound of Formula Ib:

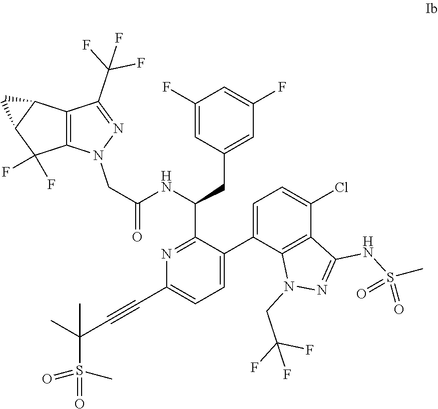 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week, and
wherein the compound of Formula Ib, or a pharmaceutically acceptable salt thereof, is administered as a monotherapy.

In some embodiments, the present disclosure provides a method of treating or preventing HIV in a patient, comprising administering to the patient a compound of Formula Ib:

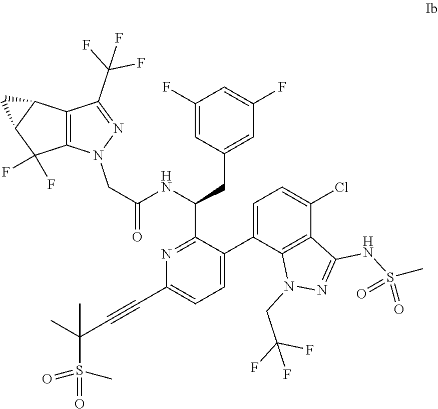 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week;
wherein if the patient misses a maintenance dosage, the method further comprises orally administering to the patient about 300 mg of the compound of Formula Ib, or a pharmaceutically acceptable salt thereof, within a third period of time which is about one day to about fourteen days after the patient first misses the maintenance dosage, and resuming administration of the one or more maintenance dosages within about 1 day to about 7 days after the oral administration during the third period of time.

In some embodiments, the present disclosure provides a method of treating or preventing HIV in a patient, comprising administering to the patient a compound of Formula Ib:

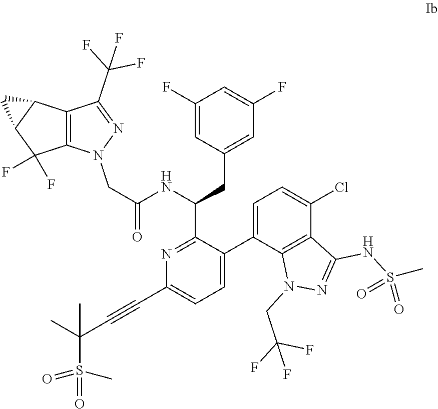 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week;
wherein if the patient misses two consecutive maintenance dosages, the method further comprises orally administering to the patient about 600 mg of the compound of Formula Ib, or a pharmaceutically acceptable salt thereof, within a third period of time which is about one day to about fourteen days after the patient first misses the second maintenance dosage, and resuming administration of the one or more maintenance dosages within about 1 day to about 7 days after the oral administrations during the third period of time.

In some embodiments, the present disclosure provides a method of preventing HIV in a patient, comprising administering to the patient a compound of Formula Ib:

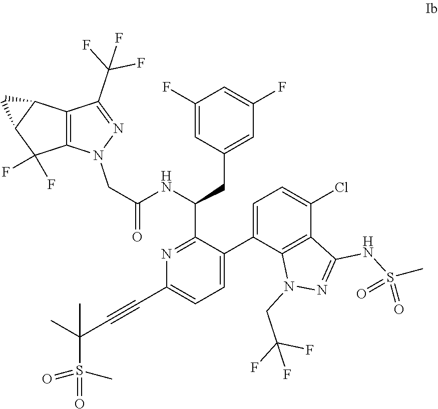 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week;
wherein the method comprises pre-exposure prophylaxis (PrEP).

In some embodiments, the present disclosure provides a method of preventing HIV in a patient, comprising administering to the patient a compound of Formula Ib:

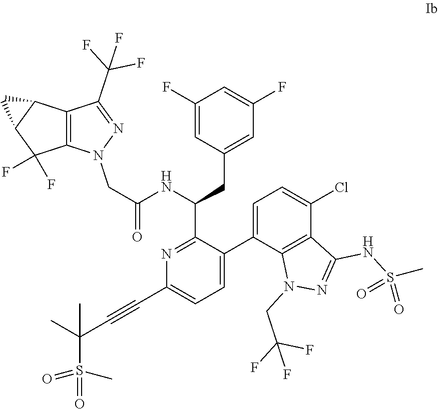 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week;
wherein if the patient misses a maintenance dosage, the method further comprises orally administering to the patient about 300 mg of the compound of Formula Ib, or a pharmaceutically acceptable salt thereof, within a third period of time which is about one day to about fourteen days after the patient first misses the maintenance dosage, and resuming administration of the one or more maintenance dosages within about 1 day to about 7 days after the oral administration during the third period of time; and wherein the method comprises pre-exposure prophylaxis (PrEP).

In some embodiments, the present disclosure provides a method of preventing HIV in a patient, comprising administering to the patient a compound of Formula Ib:

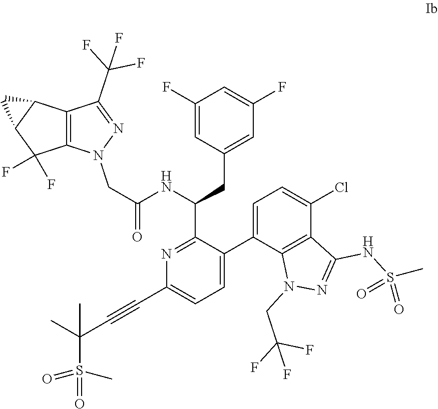 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt thereof, the method comprising:
(i) orally administering to the patient an initiation dosage comprising about 600 mg of the compound of Formula Ib, or a pharmaceutically acceptable salt thereof, for two days; and
(ii) orally administering to the patient one or more maintenance dosages each comprising about 300 mg of the compound of Formula Ib, or a pharmaceutically acceptable salt thereof, once per week;
wherein if the patient misses two consecutive maintenance dosages, the method further comprises orally administering to the patient about 600 mg of the compound of Formula Ib, or a pharmaceutically acceptable salt thereof, within a third period of time which is about one day to about fourteen days after the patient first misses the second maintenance dosage, and resuming administration of the one or more maintenance dosages within about 1 day to about 7 days after the oral administrations during the third period of time; and
wherein the method comprises pre-exposure prophylaxis (PrEP).

In some embodiments, the methods provided herein comprise preventing an human immunodeficiency virus (HIV) infection in the patient. In some embodiments, the patient may have or be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), and/or a sex worker. In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender woman; and/or
a person who injects drugs, including, for example, but not limited to people who injects opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

The term “patient” is meant to refer to a human who is in need of therapeutic or preventative treatment for a viral infection, such as HIV infection.

As used herein, the terms “prevention” or “preventing” refers to the administration of a compound, pharmaceutically acceptable salt thereof, or composition comprising the compound or the pharmaceutically acceptable salt thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The term also refers to prevention of transmission of HIV through blood transfusion.

As used herein the term “Cₜₐᵤ” refers to the observed drug concentration at the end of the dosing interval.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of the compound of Formula Ia or Ib, or a pharmaceutically acceptable salt thereof, to the patient. As used herein, the terms “event driven” or “event driven administration” refer to administration of the compound of Formula Ia or Ib, or a pharmaceutically acceptable salt thereof, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sex partners; exchange of money, gifts, shelter or drugs for anal sex; sex with male partner and diagnosis of sexually transmitted infection; and no consistent use of condoms with sex partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, the compound of Formula Ia or Ib, or a pharmaceutically acceptable salt thereof, is administered before exposure of the patient to the HIV.

In some embodiments, the compound of Formula Ia or Ib, or a pharmaceutically acceptable salt thereof, is administered during the period of exposure of the patient to the HIV.

In some embodiments, the compound of Formula Ia or Ib, or a pharmaceutically acceptable salt thereof, is administered after final exposure of the patient to the HIV.

In some embodiments, the compound of Formula Ia or Ib, or a pharmaceutically acceptable salt thereof, is administered before and during exposure of the patient to the HIV.

In some embodiments, the compound of Formula Ia or Ib, or a pharmaceutically acceptable salt thereof, is administered before and after exposure of the patient to the HIV.

In some embodiments, the compound of Formula Ia or Ib, or a pharmaceutically acceptable salt thereof, is administered during and after exposure of the patient to the HIV.

In some embodiments, the compound of Formula Ia or Ib, or a pharmaceutically acceptable salt thereof, is administered before, during, and after exposure of the patient to the HIV.

In some embodiments, the dose of the compound of Formula Ia or Ib, or a pharmaceutically acceptable salt thereof, administered during each period (i.e., before, during, and after exposure) may be different, i.e, independently selected from any of the doses disclosed herein.

In certain embodiments, e.g., when administered as PrEP, the compound of Formula Ia or Ib, or a pharmaceutically acceptable salt thereof, is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) prior to an event that would increase the patient's risk of acquiring HIV (e.g., prior to sexual intercourse or other exposure to the HIV). In some embodiments, the compound of Formula Ia or Ib, or a pharmaceutically acceptable salt thereof, is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, the compound of Formula Ia or Ib, or a pharmaceutically acceptable salt thereof, is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when the compound of Formula Ia or Ib, or a pharmaceutically acceptable salt thereof, is administered prior to an event that would increase the patient' risk of acquiring HIV, it is administered daily prior to the event (e.g., sexual activity). In certain embodiments, when the compound of Formula Ia or Ib, or a pharmaceutically acceptable salt thereof, is administered prior to an event that would increase the patient's risk of acquiring HIV, it is administered one to three times prior to the event. In certain embodiments, when the compound of Formula Ia or Ib, or a pharmaceutically acceptable salt thereof, is administered prior to an event that would increase the patient's risk of acquiring HIV, it is administered one time (i.e., once) prior to the event.

In some embodiments, the compound of Formula Ia or Ib, or a pharmaceutically acceptable salt thereof, is administered from about 14 days to about one day before exposure of the patient to the HIV. In some embodiments, the compound of Formula Ia or Ib, or a pharmaceutically acceptable salt thereof, is administered once from about 14 days to about one day before exposure of the patient to the HIV.

In some embodiments, the compound of Formula Ia or Ib, or a pharmaceutically acceptable salt thereof, is administered from about 10 days to about 5 days before exposure of the patient to the HIV. In some embodiments, the compound of Formula Ia or Ib, or a pharmaceutically acceptable salt thereof, is administered once from about 10 days to about 5 days before exposure of the patient to the HIV.

In some embodiments, the compound of Formula Ia or Ib, or a pharmaceutically acceptable salt thereof, is administered from about 8 days to about 6 days before exposure of the patient to the HIV. In some embodiments, the compound of Formula Ia or Ib, or a pharmaceutically acceptable salt thereof, is administered once from about 8 days to about 6 days before exposure of the patient to the HIV.

In some embodiments, the compound of Formula Ia or Ib, or a pharmaceutically acceptable salt thereof, is administered about 7 days before exposure of the patient to the HIV. In some embodiments, the compound of Formula Ia or Ib, or a pharmaceutically acceptable salt thereof, is administered once about 7 days before exposure of the patient to the HIV.

In some embodiments, the compound of Formula Ia or Ib, or a pharmaceutically acceptable salt thereof, is administered from about 72 hours to about 1 hour before exposure of the patient to the HIV. In some embodiments, the compound of Formula Ia or Ib, or a pharmaceutically acceptable salt thereof, is administered once from about 72 hours to about 1 hour before exposure of the patient to the HIV.

In some embodiments of the methods provided herein, the pre-exposure prophylaxis (PrEP) comprises continuous PrEP.

In certain embodiments where the compound of Formula Ia or Ib, or a pharmaceutically acceptable salt thereof, is administered before exposure of the patient to the HIV, the methods disclosed herein further comprise administering one or more additional doses of the compound of Formula Ia or Ib, or a pharmaceutically acceptable salt thereof, during, and/or after exposure of the patient to the HIV.

In some embodiments, e.g., when administered as part of a PrEP regimen or as part of a PEP regimen, the compound of Formula Ia or Ib, or a pharmaceutically acceptable salt thereof, is administered during the period of exposure of the patient to the HIV. In certain embodiments wherein the compound of Formula Ia or Ib, or a pharmaceutically acceptable salt thereof, is administered before HIV exposure, the compound of Formula Ia or Ib, or a pharmaceutically acceptable salt thereof, is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with sex partner known to be HIV positive). In some embodiments, the compound of Formula Ia or Ib, or a pharmaceutically acceptable salt thereof, is administered once about every 7 days, about every 14 days, about every 21 days, about every 28 days, about every 35 days, about every 42 days, about every 6 months, or about every 12 months during the period of exposure of the patient to the HIV.

In some embodiments, the compound of Formula Ia or Ib, or a pharmaceutically acceptable salt thereof, administered prior to exposure to the HIV is at a different dose than the compound of Formula Ia or Ib, or a pharmaceutically acceptable salt thereof, administered during and/or after exposure to the HIV. For example, in some embodiments, the dose of the compound of Formula Ia or Ib, or a pharmaceutically acceptable salt thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt thereof, is a double dose. In some embodiments, the dose of the compound of Formula Ia or Ib, or a pharmaceutically acceptable salt thereof, is decreased, e.g., a half dose as compared to an earlier administered dose (e.g., a dose prior to exposure to the HIV).

In some embodiments, the compound of Formula Ia or Ib, or a pharmaceutically acceptable salt thereof, is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in ile-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, the compound of Formula Ia or Ib, or a pharmaceutically acceptable salt thereof, is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, the compound of Formula Ia or Ib, or a pharmaceutically acceptable salt thereof, is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered for 30 days following an event that would increase the patient's risk of acquiring HIV (e.g., following sexual intercourse or other exposure to the HIV). In certain embodiments, the compound of Formula Ia or Ib, or a pharmaceutically acceptable salt thereof, is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, the compound of Formula Ia or Ib, or a pharmaceutically acceptable salt thereof, is administered for 1 day, 2 days, 3 days, 4 days, or 5 days following an event that would increase the patient's risk of acquiring HIV (e.g., following sexual intercourse or other exposure to the HIV). In certain embodiments, when the compound of Formula Ia or Ib, or a pharmaceutically acceptable salt thereof, is administered following an event that would increase the patient's risk of acquiring HIV, it is administered daily following the event. In certain embodiments, when the compound of Formula Ia or Ib, or a pharmaceutically acceptable salt thereof, is administered following an event that would increase the patient's risk of acquiring HIV, it is administered one to three times following the event. In certain embodiments, when the compound of Formula Ia or Ib, or a pharmaceutically acceptable salt thereof, is administered following an event that would increase the patient's risk of acquiring HIV, it is administered once following the event.

In certain embodiments, e.g., when administered as PEP, the compound of Formula Ia or Ib, or a pharmaceutically acceptable salt thereof, is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every week following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every month following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 2 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 3 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 4 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 5 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 6 months following an event that would increase the patient's risk of acquiring HIV (e.g., following sexual intercourse or other exposure to the HIV). In certain embodiments, e.g., when administered as PEP, the compound of Formula Ia or Ib, or a pharmaceutically acceptable salt thereof, is administered once about every 12 months following an event that would increase the patient's risk of acquiring HIV (e.g., following sexual intercourse or other exposure to the HIV).

In certain embodiments, e.g., when administered as PEP, the compound of Formula Ia or Ib, or a pharmaceutically acceptable salt thereof, is administered for one month, two months, three months, four months, five months, six months, or twelve months following an event that would increase the patient's risk of acquiring HIV (e.g., following sexual intercourse or other exposure to the HIV).

In certain embodiments, when the compound of Formula Ia or Ib, or a pharmaceutically acceptable salt thereof, is administered following an event that would increase the patient's risk of acquiring HIV, it is administered one to fifty times following the event. In certain embodiments, when the compound of Formula Ia or Ib, or a pharmaceutically acceptable salt thereof, is administered following an event that would increase the patient's risk of acquiring HIV, it is administered one to forty times following the event. In certain embodiments, when the compound of Formula Ia or Ib, or a pharmaceutically acceptable salt thereof, is administered following an event that would increase the patient's risk of acquiring HIV, it is administered one to thirty times following the event. In certain embodiments, when the compound of Formula Ia or Ib, or a pharmaceutically acceptable salt thereof, is administered following an event that would increase the patient's risk of acquiring HIV, it is administered one to twenty times following the event. In certain embodiments, when the compound of Formula Ia or Ib, or a pharmaceutically acceptable salt thereof, is administered following an event that would increase the patient's risk of acquiring HIV, it is administered one to fifteen times following the event. In certain embodiments, when the compound of Formula Ia or Ib, or a pharmaceutically acceptable salt thereof, is administered following an event that would increase the patient's risk of acquiring HIV, it is administered one to ten times following the event. In certain embodiments, when the compound of Formula Ia or Ib, or a pharmaceutically acceptable salt thereof, is administered following an event that would increase the patient's risk of acquiring HIV, it is administered one to five times following the event.

In some embodiments, the compound of Formula Ia or Ib, or a pharmaceutically acceptable salt thereof, is administered during exposure of the patient to the HIV (e.g., during a period of sexual activity with sex partner known to be HIV positive).

In some embodiments, the compound of Formula Ia or Ib, or a pharmaceutically acceptable salt thereof, is administered after exposure (e.g., after final exposure) of the patient to the HIV (e.g., after a period of sexual activity with sex partner known to be HIV positive). In some embodiments, the compound of Formula Ia or Ib, or a pharmaceutically acceptable salt thereof, is administered from about 1 hour to about 14 days after exposure (e.g., after final exposure) of the patient to the HIV. In some embodiments, the compound of Formula Ia or Ib, or a pharmaceutically acceptable salt thereof, is administered once from about 1 hour to about 14 days after exposure (e.g., after final exposure) of the patient to the HIV. In some embodiments, the compound of Formula Ia or Ib, or a pharmaceutically acceptable salt thereof, is administered from about 1 hour to about 7 days after exposure (e.g., after final exposure) of the patient to the HIV. In some embodiments, the compound of Formula Ia or Ib, or a pharmaceutically acceptable salt thereof, is administered once from about 1 hour to about 7 days after exposure (e.g., after final exposure) of the patient to the HIV. In some embodiments, the compound of Formula Ia, or Ib or a pharmaceutically acceptable salt thereof, is administered from about 1 hour to about 72 hours after exposure (e.g., after final exposure) of the patient to the HIV. In some embodiments, the compound of Formula Ia or Ib, or a pharmaceutically acceptable salt thereof, is administered once from about 1 hour to about 72 hours after exposure (e.g., after final exposure) of the patient to the HIV. In some embodiments, the compound of Formula Ia or Ib, or a pharmaceutically acceptable salt thereof, is administered from about 1 hour to about 24 hours after exposure (e.g., after final exposure) of the patient to the HIV. In some embodiments, the compound of Formula Ia or Ib, or a pharmaceutically acceptable salt thereof, is administered once from about 1 hour to about 24 hours after exposure (e.g., after final exposure) of the patient to the HIV. In some embodiments, the compound of Formula Ia or Ib, or a pharmaceutically acceptable salt thereof, is administered from about 24 hours to about 72 hours after exposure (e.g., after final exposure) of the patient to the HIV. In some embodiments, the compound of Formula Ia or Ib, or a pharmaceutically acceptable salt thereof, is administered once from about 24 hours to about 72 hours after exposure (e.g., after final exposure) of the patient to the HIV.

In some embodiments, e.g., when administered as PrEP, the compound of Formula Ia or Ib, or a pharmaceutically acceptable salt thereof, is administered prior to an event that would increase the patient's risk of acquiring HIV (e.g., prior to sexual activity), and following the event. For example, in certain embodiments, when administered as PrEP, the compound of Formula Ia or Ib, or a pharmaceutically acceptable salt thereof, is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72, 1 hour to 48 hours, 1 hour to 36 hours, 1 hour to 24 hours, or 1 hour to 12 hours following the event. For example, in some embodiments, one or more (e.g., one, two, or three) dosages of the compound of Formula Ia or Ib, or a pharmaceutically acceptable salt thereof, are administered one to ten days (e.g., seven days) prior to an event that would increase the patient's risk of acquiring HIV (e.g., prior to sexual intercourse) and once during a period of one to ten days following the event. In some embodiments, the compound of Formula Ia or Ib, or a pharmaceutically acceptable salt thereof, is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

Also provided herein is a method of reducing the risk of acquiring HIV in a patient, comprising administering to the patient a compound of Formula Ia or Ib, or a pharmaceutically acceptable salt thereof.

In some embodiments, methods for reducing the risk of acquiring HIV (e.g., HIV-1 and/or HIV-2) comprise administration of the compound of Formula Ia or Ib, or a pharmaceutically acceptable salt thereof, to a patient in combination with safer sexual intercourse practices. In certain embodiments, methods for reducing the risk of acquiring HIV (e.g., HIV-1 and/or HIV-2) comprise administration of the compound of Formula Ia or Ib, or a pharmaceutically acceptable salt thereof, to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

In some embodiments, the patient is a heavily treatment-experienced patient. In some embodiments, the methods provided herein comprise treating human immunodeficiency virus (HIV) infection in a heavily treatment-experienced patient.

In some embodiments, the present disclosure relates to the use of the compound of Formula Ia or Ib, or a pharmaceutically acceptable salt thereof, in treating an infection caused by the HIV virus comprising administering a therapeutically effective amount to a patient in need thereof, where the patient is a heavily treatment-experienced patient that has a multidrug resistant HIV infection.

As used herein, a “heavily treatment-experienced patient” refers to an HIV-infected patient who has limited treatment options due to a multidrug resistant HIV infection. For example, in some embodiments, the “heavily treatment-experienced patient” is a patient with HIV who has developed resistance to an antiretroviral medication from at least one class of antiretroviral medications selected from the group consisting of NRTIs, NNRTIs, PIs, and INSTIs.

In some embodiments, “multidrug resistant HIV infection” means resistance to an antiretroviral medication from at least one class of antiretroviral medications selected from the group consisting of NRTIs, NNRTIs, PIs, and INSTIs. In some embodiments, “multidrug resistant HIV infection” means resistance to at least one antiretroviral medication from two classes of antiretroviral medications selected from the group consisting of NRTIs, NNRTIs, PIs, and INSTIs. In some embodiments, “multidrug resistant HIV infection” means resistance to at least one antiretroviral medication from three classes of antiretroviral medications selected from the group consisting of NRTIs, NNRTIs, PIs, and INSTIs. In some embodiments, “multidrug resistant HIV infection” means resistance to at least one antiretroviral medication from each of the four classes of antiretroviral medications selected from the group consisting of NRTIs, NNRTIs, PIs, and INSTIs.

As used herein, the term “NRTI(s)” refers to nucleoside reverse transcriptase inhibitor(s) or nucleotide reverse transcriptase inhibitor(s).

As used herein, the term “NNRTI(s)” refers to non-nucleoside reverse transcriptase inhibitor(s) or non-nucleotide reverse transcriptase inhibitor(s).

As used herein, the term “PI(s)” refers to protease inhibitor(s).

As used herein, the term “INSTI(s)” refers to integrase strand transfer inhibitor(s).

As used herein, the term “fail” or “failed” when referring to HIV therapy or an HIV treatment regimen means a treatment outcome which precludes the use of the same agent or class in the future in a patient with HIV. This could be due to inadequate initial viral response due to pre-existing viral resistance, viral rebound due to emergent viral resistance, or inability of a patient to continue a treatment due to intolerability or safety issues.

In the disclosed methods, the heavily treatment-experienced patient is infected with multidrug resistant HIV. In some embodiments, the heavily treatment-experienced patient has a multidrug resistant HIV infection and is on a failing HIV treatment regimen. In some embodiments, the heavily treatment-experienced patient has a viral load greater than about 1,000 copies of HIV RNA/mL.

In some embodiments, the HIV infection is an HIV-1 infection. In some embodiments, the HIV-1 infection is characterized by HIV-1 mutant resistance to an antiretroviral medication, for example, to one, two, three, four, or more classes of antiretroviral medications (e.g., PIs, NRTIs, NNRTIs, INSTIs, etc.). In some embodiments, the HIV-1 infection is characterized by HIV-1 mutant resistance to one or more classes of antiretroviral medications. In some embodiments, the HIV-1 infection is characterized by HIV-1 mutant resistance to two or more classes of antiretroviral medications. In some embodiments, the HIV-1 infection is characterized by HIV-1 mutant resistance to three or more classes of antiretroviral medications.

In some embodiments, the HIV-1 mutant is resistant to a protease inhibitor (PI), a nucleoside or nucleotide reverse transcriptase inhibitor (NRTI), a non-nucleoside or non-nucleotide reverse transcriptase inhibitor (NNRTI), or an integrase strand transfer inhibitor (INSTI).

In some embodiments, the HIV-1 infection is characterized by an HIV-1 mutant that includes, but is not limited to:
(a) an HIV-1 mutant resistant to a PI (e.g., I50V, I84V/L90M, G48V/V82A/L90M, G48V/V82S, etc.);
(b) an HIV-1 mutant resistant to an NRTI (e.g., K65R, M184V, 6TAMs, etc.);
(c) an HIV-1 mutant resistant to an NNRTI (e.g., K103N, Y181C, Y188L, L100I/K103N, K103N/Y181C, etc.); and/or
(d) an HIV-1 mutant resistant to an INSTI (Y143R, E138K/Q148K, G140S/Q148R, E92Q/N155H, N155H/Q148R, R263K/M50I, etc.).

In some embodiments, the HIV-1 mutant is resistant to a protease inhibitor selected from I50V, I84V/L90M, G48V/V82A/L90M, and G48V/V82S.

In some embodiments, the HIV-1 mutant is resistant to a nucleoside or nucleotide reverse transcriptase inhibitor selected from K65R, M184V, and 6TAMs.

In some embodiments, the HIV-1 mutant is resistant to a non-nucleoside or non-nucleotide reverse transcriptase inhibitor selected from K103N, Y181C, Y188L, L100I/K103N, and K103N/Y181C.

In some embodiments, the HIV-1 mutant is resistant to an integrase strand transfer inhibitor selected from Y143R, E138K/Q148K, G140S/Q148R, E92Q/N155H, N155H/Q148R, and R263K/M50I.

In some embodiments, the patient is infected with HIV-1 that is resistant to at least one antiretroviral medication. In some embodiments, the patient is infected with multidrug resistant HIV-1. In some embodiments, the patient is infected with multidrug resistant HIV-1 that is resistant to at least one, two, three, four, or more antiretroviral medications. In some embodiments, the patient is infected with multidrug resistant HIV-1 that is resistant to at least one antiretroviral medication from each of two different classes of antiretroviral medications. In some embodiments, the patient is infected with multidrug resistant HIV-1 that is resistant to at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the patient is infected with multidrug resistant HIV-1 that is resistant to at least one NRTI and at least one NNRTI. In some embodiments, the patient is infected with multidrug resistant HIV-1 that is resistant to at least one NRTI and at least one PI. In some embodiments, the patient is infected with multidrug resistant HIV-1 that is resistant to at least one NRTI and at least one INSTI. In some embodiments, the patient is infected with multidrug resistant HIV-1 that is resistant to at least one NNRTI and at least one PI. In some embodiments, the patient is infected with multidrug resistant HIV-1 that is resistant to at least one NNRTI and at least one INSTI. In some embodiments, the patient is infected with multidrug resistant HIV-1 that is resistant to at least one PI and at least one INSTI. In some embodiments, the patient is infected with multidrug resistant HIV-1 that is resistant to at least one NRTI, at least one NNRTI, and at least one PI. In some embodiments, the patient is infected with multidrug resistant HIV-1 that is resistant to at least one NRTI, at least one NNRTI, and at least one INSTI. In some embodiments, the patient is infected with multidrug resistant HIV-1 that is resistant to at least one NRTI, at least one PI, and at least one INSTI. In some embodiments, the patient is infected with multidrug resistant HIV-1 that is resistant to at least one NNRTI, at least one PI, and at least one INSTI. In some embodiments, the patient is infected with multidrug resistant HIV-1 that is resistant to at least one NRTI, at least one NNRTI, at least one PI, and at least one INSTI.

In some embodiments, the patient is infected with multidrug resistant HIV-1 that is resistant to at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the patient is infected with multidrug resistant HIV-1 that is resistant to at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the patient is infected with multidrug resistant HIV-1 that is resistant to at least one antiretroviral medication that is a PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the patient is infected with multidrug resistant HIV-1 that is resistant to at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the patient is infected with multidrug resistant HIV-1 that is resistant to at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the patient is infected with multidrug resistant HIV-1 that is resistant to at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the patient has been previously treated with at least one antiretroviral medication before being treated with a compound of Formula Ia or Ib, or a pharmaceutically acceptable salt thereof. In some embodiments, the patient has been previously treated with at least one antiretroviral medication for at least 3 months, such as at least 4 months, at least 5 months, at least 6 months, at least 7 months, at least 8 months, at least 9 months, at least 10 months, at least 11 months, at least 12 months, at least 18 months, or at least 24 months. In some embodiments, the patient has been previously treated with at least one antiretroviral medication for at least 3 months. In some embodiments, the patient has been previously treated with at least one antiretroviral medication for at least 6 months. In some embodiments, the patient has been previously treated with at least one antiretroviral medication for at least 9 months. In some embodiments, the patient has been previously treated with at least one antiretroviral medication for at least 12 months. In some embodiments, the patient has been previously treated with at least one antiretroviral medication for at least 18 months. In some embodiments, the patient has been previously treated with at least one antiretroviral medication for at least 24 months. In some embodiments, the patient has been previously treated with at least one antiretroviral medication for at least 30 months. In some embodiments, the patient has been previously treated with at least one antiretroviral medication for at least 36 months.

In some embodiments of the disclosed methods, the patient has failed a prior HIV treatment regimen before being treated with a compound of Formula Ia or Ib, or a pharmaceutically acceptable salt thereof. In some embodiments of the disclosed methods, the patient is failing an HIV treatment regimen at the time of beginning administration of the compound of Formula Ia or Ib, or a pharmaceutically acceptable salt thereof. In some embodiments, the prior HIV treatment regimen included administering at least one antiretroviral medication. In some embodiments, the patient infected with HIV has relapsed after an initial response to the prior HIV treatment regimen, for example, antiretroviral therapy. In some embodiments, the patient has a viral load of greater than about 50 copies of HIV RNA/mL after about 48 weeks of therapy, for example, antiretroviral therapy, before being treated with a compound of Formula Ia or Ib, or a pharmaceutically acceptable salt thereof.

In some embodiments, the prior treatment regimen includes administering at least one antiretroviral medication from each of two different classes of antiretroviral medications. In some embodiments, the prior treatment regimen includes administering at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the prior treatment regimen includes administering at least one NRTI and at least one NNRTI. In some embodiments, the prior treatment regimen includes administering at least one NRTI and at least one PI. In some embodiments, the prior treatment regimen includes administering at least one NRTI and at least one INSTI. In some embodiments, the prior treatment regimen includes administering at least one NNRTI and at least one PI. In some embodiments, the prior treatment regimen includes administering at least one NNRTI and at least one INSTI. In some embodiments, the prior treatment regimen includes administering at least one PI and at least one INSTI. In some embodiments, the prior treatment regimen includes administering at least one NRTI, at least one NNRTI, and at least one PI. In some embodiments, the prior treatment regimen includes administering at least one NRTI, at least one NNRTI, and at least one INSTI. In some embodiments, the prior treatment regimen includes administering at least one NRTI, at least one PI, and at least one INSTI. In some embodiments, the prior treatment regimen includes administering at least one NNRTI, at least one PI, and at least one INSTI.

In some embodiments, the prior treatment regimen includes administering at least one antiretroviral medication that is a gp41 fusion inhibitor.

In some embodiments, the prior treatment regimen includes administering at least one antiretroviral medication that is a CCR5 co-receptor antagonist.

In some embodiments, the prior treatment regimen includes administering at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the prior treatment regimen includes administering at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the prior treatment regimen includes administering at least one antiretroviral medication that is a PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the prior treatment regimen includes administering at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the prior treatment regimen includes administering at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the prior treatment regimen includes administering at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the heavily treatment-experienced patient infected with HIV has a viral load of about 200 copies of HIV-1 RNA/mL (c/mL) to about 1,000,000 c/mL at the time of beginning administration of the compound of Formula Ia or Ib, or a pharmaceutically acceptable salt thereof, such as a viral load of about 200 c/mL to about 500,000 c/mL, about 200 c/mL to about 250,000 c/mL, about 200 c/mL to about 100,000 c/mL, about 200 c/mL to about 50,000 c/mL, about 200 c/mL to about 25,000 c/mL, about 200 c/mL to about 10,000 c/mL, about 200 c/mL to about 5,000 c/mL, about 200 c/mL to about 3,000 c/mL, about 200 c/mL to about 2,000 c/mL, about 200 c/mL to about 1,000 c/mL, about 200 c/mL to about 750 c/mL, about 200 c/mL to about 500 c/mL, about 500 c/mL to about 1,000,000 c/mL, about 500 c/mL to about 500,000 c/mL, about 500 c/mL to about 250,000 c/mL, about 500 c/mL to about 100,000 c/mL, about 500 c/mL to about 50,000 c/mL, about 500 c/mL to about 25,000 c/mL, about 500 c/mL to about 10,000 c/mL, about 500 c/mL to about 5,000 c/mL, about 500 c/mL to about 3,000 c/mL, about 500 c/mL to about 2,000 c/mL, about 500 c/mL to about 1,000 c/mL, about 500 c/mL to about 750 c/mL, about 750 c/mL to about 1,000,000 c/mL, about 750 c/mL to about 500,000 c/mL, about 750 c/mL to about 250,000 c/mL, about 750 c/mL to about 100,000 c/mL, about 750 c/mL to about 50,000 c/mL, about 750 c/mL to about 25,000 c/mL, about 750 c/mL to about 10,000 c/mL, about 750 c/mL to about 5,000 c/mL, about 750 c/mL to about 3,000 c/mL, about 750 c/mL to about 2,000 c/mL, about 750 c/mL to about 1,000 c/mL, about 1,000 c/mL to about 1,000,000 c/mL, about 1,000 c/mL to about 500,000 c/mL, about 1,000 c/mL to about 250,000 c/mL, about 1,000 c/mL to about 100,000 c/mL, about 1,000 c/mL to about 50,000 c/mL, about 1,000 c/mL to about 25,000 c/mL, about 1,000 c/mL to about 10,000 c/mL, about 1,000 c/mL to about 5,000 c/mL, about 1,000 c/mL to about 3,000 c/mL, about 1,000 c/mL to about 2,000 c/mL, about 2,000 c/mL to about 1,000,000 c/mL, about 2,000 c/mL to about 500,000 c/mL, about 2,000 c/mL to about 250,000 c/mL, about 2,000 c/mL to about 100,000 c/mL, about 2,000 c/mL to about 50,000 c/mL, about 2,000 c/mL to about 25,000 c/mL, about 2,000 c/mL to about 10,000 c/mL, about 2,000 c/mL to about 5,000 c/mL, about 2,000 c/mL to about 3,000 c/mL, about 3,000 c/mL to about 1,000,000 c/mL, about 3,000 c/mL to about 500,000 c/mL, about 3,000 c/mL to about 250,000 c/mL, about 3,000 c/mL to about 100,000 c/mL, about 3,000 c/mL to about 50,000 c/mL, about 3,000 c/mL to about 25,000 c/mL, about 3,000 c/mL to about 10,000 c/mL, about 3,000 c/mL to about 5,000 c/mL, about 5,000 c/mL to about 1,000,000 c/mL, about 5,000 c/mL to about 500,000 c/mL, about 5,000 c/mL to about 250,000 c/mL, about 5,000 c/mL to about 100,000 c/mL, about 5,000 c/mL to about 50,000 c/mL, about 5,000 c/mL to about 25,000 c/mL, about 5,000 c/mL to about 10,000 c/mL, about 10,000 c/mL to about 1,000,000 c/mL, about 10,000 c/mL to about 500,000 c/mL, about 10,000 c/mL to about 250,000 c/mL, about 10,000 c/mL to about 100,000 c/mL, about 10,000 c/mL to about 50,000 c/mL, about 10,000 c/mL to about 25,000 c/mL, about 25,000 c/mL to about 1,000,000 c/mL, about 25,000 c/mL to about 500,000 c/mL, about 25,000 c/mL to about 250,000 c/mL, about 25,000 c/mL to about 100,000 c/mL, about 25,000 c/mL to about 50,000 c/mL, about 50,000 c/mL to about 1,000,000 c/mL, about 50,000 c/mL to about 500,000 c/mL, about 50,000 c/mL to about 250,000 c/mL, about 50,000 c/mL to about 100,000 c/mL, about 100,000 c/mL to about 1,000,000 c/mL, about 100,000 c/mL to about 500,000 c/mL, about 100,000 c/mL to about 250,000 c/mL, about 250,000 c/mL to about 1,000,000 c/mL, about 250,000 c/mL to about 500,000 c/mL, or about 500,000 c/mL to about 1,000,000 c/mL.

In some embodiments, the patient has a viral load of greater than about 200 copies of HIV-1 RNA/mL (c/mL) at the time of beginning administration of the compound of Formula Ia or Ib, or a pharmaceutically acceptable salt thereof, such as a viral load greater than about 500 c/mL, about 750 c/mL, about 1,000 c/mL, about 2,000 c/mL, about 3,000 c/mL, about 5,000 c/mL, about 10,000 c/mL, about 25,000 c/mL, about 50,000 c/mL, about 100,000 c/mL, about 250,000 c/mL, about 500,000 c/mL, or greater than about 1,000,000 c/mL at the time of beginning administration of the compound of Formula Ia or Ib, or a pharmaceutically acceptable salt thereof. In some embodiments, the patient has a viral load of greater than about 200 c/mL at the time of beginning administration of the compound of Formula Ia or Ib, or a pharmaceutically acceptable salt thereof. In some embodiments, the patient has a viral load of greater than about 500 c/mL at the time of beginning administration of the compound of Formula Ia or Ib, or a pharmaceutically acceptable salt thereof. In some embodiments, the patient has a viral load of greater than about 750 c/mL at the time of beginning administration of the compound of Formula Ia or Ib, or a pharmaceutically acceptable salt thereof. In some embodiments, the patient has a viral load of greater than about 1,000 c/mL at the time of beginning administration of the compound of Formula Ia or Ib, or a pharmaceutically acceptable salt thereof. In some embodiments, the patient has a viral load of greater than about 2,000 c/mL at the time of beginning administration of the compound of Formula Ia or Ib, or a pharmaceutically acceptable salt thereof.

In some embodiments of the disclosed methods, administration of the compound of Formula Ia or Ib, or a pharmaceutically acceptable salt thereof, results in a decrease in the viral load in the patient. In some embodiments, the viral load is decreased by about 0.5 log₁₀ to about 2.5 log₁₀ after administration of the compound of Formula Ia or Ib, or a pharmaceutically acceptable salt thereof, for a certain amount of time as compared to the viral load at the time of beginning administration of the compound of Formula Ia or Ib, or a pharmaceutically acceptable salt thereof. For example, the viral load is decreased by about 0.5 log₁₀, about 1 log₁₀, about 1.5 log₁₀, about 2 log₁₀, or about 2.5 log₁₀ after administration of the compound of Formula Ia or Ib, or a pharmaceutically acceptable salt thereof, for a certain amount of time as compared to the viral load at the time of beginning administration of the compound of Formula Ia or Ib, or a pharmaceutically acceptable salt thereof. In some embodiments, the viral load is decreased by about 0.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 1 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 1.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 2 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof. In some embodiments, the viral load is decreased by about 2.5 log₁₀ after administration of the compound of Formula Ia or Ib, or a pharmaceutically acceptable salt thereof, for about 24 weeks as compared to the viral load at the time of beginning administration of the compound of Formula Ia or Ib, or a pharmaceutically acceptable salt thereof.

In some embodiments of the disclosed methods, the viral load in the patient is about 200 c/mL or less after administration of the compound of Formula Ia or Ib, or a pharmaceutically acceptable salt thereof, for a certain amount of time, such as about 175 c/mL or less, about 150 c/mL or less, about 125 c/mL or less, about 100 c/mL or less, about 75 c/mL or less, or about 50 c/mL or less. In some embodiments, the viral load in the patient is about 200 c/mL or less after administration of the compound of Formula Ia or Ib, or a pharmaceutically acceptable salt thereof, for about 24 weeks. In some embodiments, the viral load in the patient is about 200 c/mL or less after administration of the compound of Formula Ia or Ib, or a pharmaceutically acceptable salt thereof, for about 24 weeks. In some embodiments, the viral load in the patient is about 100 c/mL or less after administration of the compound of Formula Ia or Ib, or a pharmaceutically acceptable salt thereof, for about 24 weeks. In some embodiments, the viral load in the patient is about 50 c/mL or less after administration of the compound of Formula Ia or Ib, or a pharmaceutically acceptable salt thereof, for about 24 weeks.

In certain embodiments of the disclosed methods, the heavily treatment-experienced patient is concurrently being treated with at least one additional antiretroviral medication. In some embodiments, the antiretroviral medication is selected from an NRTI, an NNRTI, a PI, an INSTI, a gp41 fusion inhibitor, and a CCR5 co-receptor antagonist.

In some embodiments, the patient is concurrently being treated with at least one NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the patient is concurrently being treated with at least one NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the patient is concurrently being treated with at least one PI. Examples of PIs include, but are not limited to, amprenavir (Agenerase®), atazanavir (Reyataz®), darunavir (Prezista®), fosamprenavir (Telzir®, Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the patient is concurrently being treated with at least one INSTI. Examples of INSTIs include, but are not limited to, raltegravir (Isentress®), elvitegravir (Vitekta®), dolutegravir (Tivicay®), cabortegravir, and bictegravir.

In some embodiments, the patient is concurrently being treated with at least one gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer, and sifuvirtide.

In some embodiments, the patient is concurrently being treated with at least one CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

Also provided in this disclosure is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication and had failed the treatment regimen. In some embodiments, the HIV treatment regimen includes administration of at least one antiretroviral medication such as those described herein. In some embodiments of the method, administration of the compound or Formula Ia or Ib, or a pharmaceutically acceptable salt thereof, results in a reduction in HIV viral load in the patient.

Also disclosed is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication and had failed the treatment regimen, where the multidrug resistant HIV-1 is resistant to at least one antiretroviral medication from each of two different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compound of Formula Ia or Ib, or a pharmaceutically acceptable salt thereof, and administration of the compound results in a reduction in HIV viral load in the patient.

In some embodiments, disclosed is a method of treating an HIV-1 infection in a heavily treatment-experienced patient with multidrug resistant HIV-1 that includes administering a therapeutically effective amount of a compound of Formula Ia or Ib, or a pharmaceutically acceptable salt thereof, to a patient that had been previously treated with an HIV treatment regimen that includes the administration of at least one antiretroviral medication, and had failed the treatment regimen, where the multidrug resistant HIV-1 is resistant to at least one antiretroviral medication from each of three different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compound of Formula Ia or Ib, or a pharmaceutically acceptable salt thereof, and administration of the compound results in a reduction in HIV viral load in the patient.

### Salts and Compositions

The present methods comprise administration of salts of the compound of Formula Ia or Ib, such as pharmaceutically acceptable salts. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. In some embodiments, the salt is a sodium salt.

The compound of Formula Ia or Ib, or a salt thereof, can be present in a composition (such as a pharmaceutical composition or formulation) where the composition includes at least one compound other than the compound of Formula Ia or Ib or a salt thereof.

In some embodiments, the composition comprises the compound of Formula Ia or Ib, or a salt thereof, and one or more additional compounds (e.g., one or more additional therapeutic compounds), or salts thereof. In some embodiments, the composition comprises the compound of Formula Ia or Ib, or a salt thereof; bictegravir, or a salt thereof; and one or more additional compounds (e.g., one or more additional therapeutic compounds such as tenofovir alafenamide, or a pharmaceutically acceptable salt thereof), or salts thereof.

Compositions can include mixtures containing the compound of Formula Ia or Ib, or salt thereof, and one or more solvents, substrates, carriers, etc. In some embodiments, the composition comprises the compound of Formula Ia or Ib, or salt thereof, in an amount greater than about 25% by weight, for example, greater than about 25% by weight, greater than about 50% by weight, greater than about 75% by weight, greater than about 80% by weight, greater than about 90% by weight, or greater than about 95% by weight.

The present disclosure further includes pharmaceutical compositions comprising the compound of Formula Ia or Ib, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Administration of the compound of Formula Ia or Ib, or a pharmaceutically acceptable salt thereof, can be carried out via any of the accepted modes of administration of agents for serving similar utilities. The pharmaceutical compositions of the disclosure can be prepared by combining the compound of Formula Ia or Ib, or a pharmaceutically acceptable salt thereof, with an appropriate pharmaceutically acceptable carrier and, in specific embodiments, are formulated into preparations in solid, semi solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. In some embodiments, pharmaceutical compositions of the disclosure are tablets. Pharmaceutical compositions of the disclosure are formulated so as to allow the active ingredients contained therein to be bioavailable upon administration of the composition to a patient. Compositions that will be administered to a patient take the form of one or more dosage units, where for example, a tablet may be a single dosage unit, and a container of the compound of Formula Ia or Ib, or a pharmaceutically acceptable salt thereof, in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the compound of Formula Ia or Ib, or a pharmaceutically acceptable salt thereof, for prevention of an HIV infection or reducing the risk of acquiring HIV, as described herein.

In some embodiments, the compound of Formula Ia or Ib, or a pharmaceutically acceptable salt thereof, is administered orally.

In certain embodiments, the active ingredient (for example, a compound of Formula Ia or Ib) is present as a free acid. In certain embodiments, the active ingredient (for example, a compound of Formula Ia or Ib) is present as a sodium salt. In some embodiments, the active ingredient is a compound of Formula Ia. In some embodiments, the active ingredient is a compound of Formula Ib.

In some embodiments, the compound of Formula Ia or Ib, or a pharmaceutically acceptable salt thereof, is administered orally.

In certain embodiments, an oral formulation of a compound of Formula Ia or Ib, or a pharmaceutically acceptable salt thereof, and at least one excipient is provided. Excipients can include ethanol, medium chain triglycerides, vitamin E d-a-tocopheryl polyethylene glycol 1000 succinate (TPGS), glycerol monocaprylocaprate, glycerin, and/or pharmaceutically acceptable oils. Examples of suitable medium chain triglycerides include, but are not limited to, MIGLYOL 810, MIGLYOL 821, and MIGLYOL 840. Examples of suitable pharmaceutically acceptable oils include, but are not limited to, sesame oil, castor oil, safflower oil, vegetable oil, and soybean oil. Oral formulations can include any combination of one or more suitable excipients. Excipients taken together can be present in greater than about 65% by weight of the total oral formulation, greater than about 70% by weight of the total oral formulation, greater than about 80% by weight of the total oral formulation, greater than about 90% by weight of the total oral formulation, or greater than about 95% by weight of the total oral formulation.

In some embodiments, the oral formulation of a compound of Formula Ia or Ib, or a pharmaceutically acceptable salt thereof, and at least one excipient as is prepared in hard or soft capsules. In some embodiments, the hard or soft capsules comprise a compound of Formula Ia or Ib, or a pharmaceutically acceptable salt thereof, and one or more excipients selected from the group consisting of ethanol, propylene glycol, glycerine, d-α-tocopheryl polyethylene glycol 1000 succinate (TPGS), polyoxyl 35 castor oil (e.g., Kolliphor® EL), glycerol monocapryolocaprate (e.g., Capmul® MCM), PEG 400 caprylic/capric glycerides (e.g., Labrasol®), PEG 400, diethylene glycol monoethyl ether (e.g., Transcutol®), propylene glycol monocaprylate, Type II (e.g., Capryol® 90), glyceryl monooleate, Type 40 (e.g., Peceol™) medium chain triglycerides (e.g., Miglyol® 812N), glyceryl monolinoleate (e.g., Maisine®), and polysorbate 80. In some embodiments, the hard or soft capsules comprise a compound of Formula Ia or Ib, or a pharmaceutically acceptable salt thereof, and one or more excipients selected from the group consisting of ethanol, medium chain triglycerides (e.g., Miglyol® 812N), d-α-tocopheryl polyethylene glycol 1000 succinate (TPGS), glycerol monocapryolocaprate (e.g., Capmul® MCM), PEG 400 caprylic/capric glycerides (e.g., Labrasol®), and PEG 400.

In some embodiments, the hard or soft capsules further comprise a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. Plasticizers, opacifiers, and colorants are well-known in the art. In some embodiments, the capsule shell comprises one or more pharmaceutically acceptable excipients selected from the group consisting of gelatin, glycerin, titanium dioxide, and iron oxide. In some embodiments, the iron oxide comprises iron oxide (yellow).

In some embodiments, an oral formulation of a compound of Formula Ia or Ib, or a pharmaceutically acceptable salt thereof, is provided. In certain embodiments, the oral formulation comprises a compound of Formula Ia or Ib, or a pharmaceutically acceptable salt thereof, and glycerol monocaprylocaprate. In certain embodiments, the oral formulation includes a compound of Formula Ia or Ib, or a pharmaceutically acceptable salt thereof, about 5% to about 20% ethanol, about 10% to about 30% vitamin E TPGS, and about 50% to about 85% MIGLYOL 812. In some embodiments, the oral formulation includes a compound of Formula Ia or Ib, or a pharmaceutically acceptable salt thereof, about 8% to about 15% ethanol, about 15% to about 25% vitamin E TPGS, and about 60% to about 77% MIGLYOL 812. In certain embodiments, the oral formulation includes a compound of Formula Ia or Ib, or a pharmaceutically acceptable salt thereof, in about 10% ethanol, about 20% vitamin E TPGS, and about 70% MIGLYOL 812. In certain embodiments, the oral formulation is prepared in hard gelatin capsules. In certain embodiments, the oral formulation is prepared in soft gelatin capsules.

In some embodiments, the hard gelatin or soft gelatin capsule comprises a compound of Formula Ia or Ib, or a pharmaceutically acceptable salt thereof, and a pharmaceutically acceptable excipient. In some embodiments, the hard gelatin or soft gelatin capsule comprises a compound of Formula Ia or Ib, or a pharmaceutically acceptable salt thereof, and glycerol monocaprylocaprate. In some embodiments, the concentration of a compound of Formula Ia or Ib, or a pharmaceutically acceptable salt thereof, in the hard gelatin or soft gelatin capsule is about 10 mg/ml to about 500 mg/ml. In some embodiments, the concentration of a compound of Formula Ia or Ib, or a pharmaceutically acceptable salt thereof, in the hard gelatin or soft gelatin capsule is about 10 mg/ml to about 400 mg/ml. In some embodiments, the concentration of a compound of Formula Ia or Ib, or a pharmaceutically acceptable salt thereof, in the hard gelatin or soft gelatin capsule is about 50 mg/ml to about 300 mg/ml. In some embodiments, the concentration of a compound of Formula Ia or Ib, or a pharmaceutically acceptable salt thereof, in the hard gelatin or soft gelatin capsule is about 50 mg/ml to about 200 mg/ml. In some embodiments, the concentration of a compound of Formula Ia or Ib, or a pharmaceutically acceptable salt thereof, in the hard gelatin or soft gelatin capsule is about 50 mg/ml to about 100 mg/ml. In some embodiments, the concentration of a compound of Formula Ia or Ib, or a pharmaceutically acceptable salt thereof, in the hard gelatin or soft gelatin capsule is about 10 mg/ml, about 15 mg/ml, about 20 mg/ml, about 25 mg/ml, about 30 mg/ml, about 30 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, about 200 mg/ml, 205 mg/ml, about 210 mg/ml, about 215 mg/ml, about 220 mg/ml, about 225 mg/ml, about 230 mg/ml, about 235 mg/ml, about 240 mg/ml, about 245 mg/ml, about 250 mg/ml, about 255 mg/ml, about 260 mg/ml, about 265 mg/ml, about 270 mg/ml, about 275 mg/ml, about 280 mg/ml, about 285 mg/ml, about 290 mg/ml, about 295 mg/ml, about 300 mg/ml, about 305 mg/ml, about 310 mg/ml, about 315 mg/ml, about 320 mg/ml, about 325 mg/ml, about 330 mg/ml, about 335 mg/ml, about 340 mg/ml, about 345 mg/ml, about 350 mg/ml, about 355 mg/ml, about 360 mg/ml, about 365 mg/ml, about 370 mg/ml, about 375 mg/ml, about 380 mg/ml, about 385 mg/ml, about 390 mg/ml, about 395 mg/ml, about 400 mg/ml, about 405 mg/ml, about 410 mg/ml, about 415 mg/ml, about 420 mg/ml, about 425 mg/ml, about 430 mg/ml, about 435 mg/ml, about 440 mg/ml, about 445 mg/ml, about 450 mg/ml, about 455 mg/ml, about 460 mg/ml, about 465 mg/ml, about 470 mg/ml, about 475 mg/ml, about 480 mg/ml, about 485 mg/ml, about 490 mg/ml, about 495 mg/ml, or about 500 mg/ml. In some embodiments, the concentration of a compound of Formula Ia or Ib, or a pharmaceutically acceptable salt thereof, in the hard gelatin or soft gelatin capsule is about 10 mg/ml. In some embodiments, the concentration of a compound of Formula Ia or Ib, or a pharmaceutically acceptable salt thereof, in the hard gelatin or soft gelatin capsule is about 20 mg/ml. In some embodiments, the concentration of a compound of Formula Ia or Ib, or a pharmaceutically acceptable salt thereof, in the hard gelatin or soft gelatin capsule is about 30 mg/ml. In some embodiments, the concentration of a compound of Formula Ia or Ib, or a pharmaceutically acceptable salt thereof, in the hard gelatin or soft gelatin capsule is about 40 mg/ml. In some embodiments, the concentration of a compound of Formula Ia or Ib, or a pharmaceutically acceptable salt thereof, in the hard gelatin or soft gelatin capsule is about 50 mg/ml. In some embodiments, the concentration of a compound of Formula Ia or Ib, or a pharmaceutically acceptable salt thereof, in the hard gelatin or soft gelatin capsule is about 75 mg/ml. In some embodiments, the concentration of a compound of Formula Ia or Ib, or a pharmaceutically acceptable salt thereof, in the hard gelatin or soft gelatin capsule is about 100 mg/ml. In some embodiments, the concentration of a compound of Formula Ia or Ib, or a pharmaceutically acceptable salt thereof, in the hard gelatin or soft gelatin capsule is about 125 mg/ml. In some embodiments, the concentration of a compound of Formula Ia or Ib, or a pharmaceutically acceptable salt thereof, in the hard gelatin or soft gelatin capsule is about 150 mg/ml. In some embodiments, the concentration of a compound of Formula Ia or Ib, or a pharmaceutically acceptable salt thereof, in the hard gelatin or soft gelatin capsule is about 175 mg/ml. In some embodiments, the concentration of a compound of Formula Ia or Ib, or a pharmaceutically acceptable salt thereof, in the hard gelatin or soft gelatin capsule is about 200 mg/ml. In some embodiments, the concentration of a compound of Formula Ia or Ib, or a pharmaceutically acceptable salt thereof, in the hard gelatin or soft gelatin capsule is about 225 mg/ml. In some embodiments, the concentration of a compound of Formula Ia or Ib, or a pharmaceutically acceptable salt thereof, in the hard gelatin or soft gelatin capsule is about 250 mg/ml. In some embodiments, the concentration of a compound of Formula Ia or Ib, or a pharmaceutically acceptable salt thereof, in the hard gelatin or soft gelatin capsule is about 275 mg/ml. In some embodiments, the concentration of a compound of Formula Ia or Ib, or a pharmaceutically acceptable salt thereof, in the hard gelatin or soft gelatin capsule is about 300 mg/ml. In some embodiments, the concentration of a compound of Formula Ia or Ib, or a pharmaceutically acceptable salt thereof, in the hard gelatin or soft gelatin capsule is about 325 mg/ml. In some embodiments, the concentration of a compound of Formula Ia or Ib, or a pharmaceutically acceptable salt thereof, in the hard gelatin or soft gelatin capsule is about 350 mg/ml. In some embodiments, the concentration of a compound of Formula Ia or Ib, or a pharmaceutically acceptable salt thereof, in the hard gelatin or soft gelatin capsule is about 375 mg/ml. In some embodiments, the concentration of a compound of Formula Ia or Ib, or a pharmaceutically acceptable salt thereof, in the hard gelatin or soft gelatin capsule is about 400 mg/ml. In some embodiments, the concentration of a compound of Formula Ia or Ib, or a pharmaceutically acceptable salt thereof, in the hard gelatin or soft gelatin capsule is about 425 mg/ml. In some embodiments, the concentration of a compound of Formula Ia or Ib, or a pharmaceutically acceptable salt thereof, in the hard gelatin or soft gelatin capsule is about 450 mg/ml. In some embodiments, the concentration of a compound of Formula Ia or Ib, or a pharmaceutically acceptable salt thereof, in the hard gelatin or soft gelatin capsule is about 475 mg/ml. In some embodiments, the concentration of a compound of Formula Ia or Ib, or a pharmaceutically acceptable salt thereof, in the hard gelatin or soft gelatin capsule is about 500 mg/ml.

In some embodiments, the amount of glycerol monocaprylocaprate in the hard gelatin or soft gelatin capsule comprising a compound of Formula Ia or Ib and glycerol monocaprylocaprate is about 30 w/w % to about 85 w/w %. In some embodiments, the amount of glycerol monocaprylocaprate in the hard gelatin or soft gelatin capsule comprising a compound of Formula Ia or Ib and glycerol monocaprylocaprate is about 40 w/w % to about 80 w/w %. In some embodiments, the amount of glycerol monocaprylocaprate in the hard gelatin or soft gelatin capsule comprising a compound of Formula Ia or Ib and glycerol monocaprylocaprate is about 50 w/w % to about 80 w/w %. In some embodiments, the amount of glycerol monocaprylocaprate in the hard gelatin or soft gelatin capsule comprising a compound of Formula Ia or Ib and glycerol monocaprylocaprate is about 60 w/w % to about 70 w/w %. In some embodiments, the amount of glycerol monocaprylocaprate in the hard gelatin or soft gelatin capsule comprising a compound of Formula Ia or Ib and glycerol monocaprylocaprate is about 65.9 w/w %. In some embodiments, the amount of glycerol monocaprylocaprate in the hard gelatin or soft gelatin capsule comprising a compound of Formula Ia or Ib and glycerol monocaprylocaprate is about 65.94 w/w %.

In some embodiments, the amount of the compound of Formula Ia or Ib in the hard gelatin or soft gelatin capsule comprising a compound of Formula Ia or Ib and glycerol monocaprylocaprate is about 1 w/w % to about 40 w/w %. In some embodiments, the amount of the compound of Formula Ia or Ib in the hard gelatin or soft gelatin capsule comprising a compound of Formula Ia or Ib and glycerol monocaprylocaprate is about 1 w/w % to about 35 w/w %. In some embodiments, the amount of the compound of Formula Ia or Ib in the hard gelatin or soft gelatin capsule comprising a compound of Formula Ia or Ib and glycerol monocaprylocaprate is about 2 w/w % to about 30 w/w %. In some embodiments, the amount of the compound of Formula Ia or Ib in the hard gelatin or soft gelatin capsule comprising a compound of Formula Ia or Ib and glycerol monocaprylocaprate is about 3 w/w % to about 28 w/w %. In some embodiments, the amount of the compound of Formula Ia or Ib in the hard gelatin or soft gelatin capsule comprising a compound of Formula Ia or Ib and glycerol monocaprylocaprate is about 3.4 w/w %. In some embodiments, the amount of the compound of Formula Ia or Ib in the hard gelatin or soft gelatin capsule comprising a compound of Formula Ia or Ib and glycerol monocaprylocaprate is about 3.42 w/w %.

In some embodiments, the hard gelatin or soft gelatin capsule comprising a compound of Formula Ia or Ib and glycerol monocaprylocaprate further comprises a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. In some embodiments, the hard gelatin or soft gelatin capsule comprising a compound of Formula Ia or Ib and glycerol monocaprylocaprate further comprises one or more of a pharmaceutically acceptable excipient selected from the group consisting of gelatin, glycerin, titanium dioxide, and iron oxide. In some embodiments, the hard gelatin or soft gelatin capsule comprising a compound of Formula Ia or Ib and glycerol monocaprylocaprate further comprises gelatin. In some embodiments, the hard gelatin or soft gelatin capsule comprising a compound of Formula Ia or Ib and glycerol monocaprylocaprate further comprises glycerin. In some embodiments, the hard gelatin or soft gelatin capsule comprising a compound of Formula Ia or Ib and glycerol monocaprylocaprate further comprises titanium dioxide. In some embodiments, the hard gelatin or soft gelatin capsule comprising a compound of Formula Ia or Ib and glycerol monocaprylocaprate further comprises iron oxide. In some embodiments, the hard gelatin or soft gelatin capsule comprising a compound of Formula Ia or Ib and glycerol monocaprylocaprate further comprises gelatin and glycerin. In some embodiments, the hard gelatin or soft gelatin capsule comprising a compound of Formula Ia or Ib and glycerol monocaprylocaprate further comprises gelatin, glycerin, and titanium dioxide. In some embodiments, the hard gelatin or soft gelatin capsule comprising a compound of Formula Ia or Ib and glycerol monocaprylocaprate further comprises gelatin, glycerin, titanium dioxide, and iron oxide. In some embodiments, the iron oxide of the hard gelatin or soft gelatin capsule comprising a compound of Formula Ia or Ib, glycerol monocaprylocaprate, gelatin, glycerin, titanium dioxide, and iron oxide comprises iron oxide (yellow).

In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 10 w/w % to about 40 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 10 w/w % to about 30 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 15 w/w % to about 25 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 18 w/w % to about 22 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 19.6 w/w %. In some embodiments, the amount of gelatin in the hard gelatin or soft gelatin capsule comprising a compound of Formula Ia or Ib, glycerol monocaprylocaprate, gelatin, glycerin, titanium dioxide, and iron oxide is about 19.60 w/w %.

In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 3 w/w % to about 25 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 5 w/w % to about 20 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 5 w/w % to about 15 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 8 w/w % to about 12 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 10.8 w/w %. In some embodiments, the amount of glycerin in the hard gelatin or soft gelatin capsule comprising a compound of Formula Ia or Ib, glycerol monocaprylocaprate, gelatin, glycerin, titanium dioxide, and iron oxide is about 10.80 w/w %.

In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 2 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.05 w/w % to about 1.5 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.1 w/w % to about 1.0 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.1 w/w % to about 0.5 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.2 w/w %. In some embodiments, the amount of titanium dioxide in the hard gelatin or soft gelatin capsule comprising a compound of Formula Ia or Ib, glycerol monocaprylocaprate, gelatin, glycerin, titanium dioxide, and iron oxide is about 0.22 w/w %.

In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 1 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.5 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.15 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.01 w/w % to about 0.1 w/w %. In some embodiments, the amount of iron oxide in the hard gelatin or soft gelatin capsule comprising a compound of Formula Ia or Ib, glycerol monocaprylocaprate, gelatin, glycerin, titanium dioxide, and iron oxide is about 0.02 w/w %.

In some embodiments, the hard gelatin or soft capsule comprises about 30 w/w % to about 85 w/w % of glycerol monocaprylocaprate and about 1 w/w % to about 40 w/w % of a compound of Formula Ia or Ib. In some embodiments, the hard gelatin or soft capsule comprises about 40 w/w % to about 80 w/w % of glycerol monocaprylocaprate and about 1 w/w % to about 35 w/w % of a compound of Formula Ia or Ib. In some embodiments, the hard gelatin or soft capsule comprises about 50 w/w % to about 80 w/w % of glycerol monocaprylocaprate and about 2 w/w % to about 30 w/w % of a compound of Formula Ia or Ib. In some embodiments, the hard gelatin or soft capsule comprises about 60 w/w % to about 70 w/w % of glycerol monocaprylocaprate and about 3 w/w % to about 28 w/w % of a compound of Formula Ia or Ib. In some embodiments, the hard gelatin or soft capsule comprises about 65.9 w/w % of glycerol monocaprylocaprate and about 3.4 w/w % of a compound of Formula Ia or Ib. In some embodiments, the hard gelatin or soft capsule comprises about 65.94 w/w % of glycerol monocaprylocaprate and about 3.42 w/w % of a compound of Formula Ia or Ib.

In some embodiments, the hard gelatin or soft capsule comprises about 30 w/w % to about 85 w/w % of glycerol monocaprylocaprate, about 1 w/w % to about 40 w/w % of a compound of Formula Ia or Ib, about 10 w/w % to about 40 w/w % of gelatin, about 3 w/w % to about 25 w/w % of glycerin, about 0.01 w/w % to about 2 w/w % of titanium dioxide, and about 0.01 w/w % to about 1 w/w % of iron oxide. In some embodiments, the hard gelatin or soft capsule comprises about 40 w/w % to about 80 w/w % of glycerol monocaprylocaprate, about 1 w/w % to about 35 w/w % of a compound of Formula Ia or Ib, about 10 w/w % to about 30 w/w % of gelatin, about 5 w/w % to about 20 w/w % of glycerin, about 0.05 w/w % to about 1.5 w/w % of titanium dioxide, and about 0.01 w/w % to about 0.5 w/w % of iron oxide. In some embodiments, the hard gelatin or soft capsule comprises about 50 w/w % to about 80 w/w % of glycerol monocaprylocaprate, about 2 w/w % to about 30 w/w % of a compound of Formula Ia or Ib, about 15 w/w % to about 25 w/w % of gelatin, about 5 w/w % to about 15 w/w % of glycerin, about 0.1 w/w % to about 1.0 w/w % of titanium dioxide, and about 0.01 w/w % to about 0.15 w/w % of iron oxide. In some embodiments, the hard gelatin or soft capsule comprises about 60 w/w % to about 70 w/w % of glycerol monocaprylocaprate, about 3 w/w % to about 28 w/w % of a compound of Formula Ia or Ib, about 18 w/w % to about 22 w/w % of gelatin, about 8 w/w % to about 12 w/w % of glycerin, about 0.1 w/w % to about 0.5 w/w % of titanium dioxide, and about 0.01 w/w % to about 0.1 w/w % of iron oxide. In some embodiments, the hard gelatin or soft capsule comprises about 65.9 w/w % of glycerol monocaprylocaprate, about 3.4 w/w % of a compound of Formula Ia or Ib, about 19.6 w/w % of gelatin, about 10.8 w/w % of glycerin, about 0.2 w/w % of titanium dioxide, and about 0.02 w/w % of iron oxide. In some embodiments, the hard gelatin or soft capsule comprises about 65.94 w/w % of glycerol monocaprylocaprate, about 3.42 w/w % of a compound of Formula Ia or Ib, about 19.60 w/w % of gelatin, about 10.80 w/w % of glycerin, about 0.22 w/w % of titanium dioxide, and about 0.02 w/w % of iron oxide.

In some embodiments, the iron oxide in the hard gelatin or soft gelatin capsule comprising a compound of Formula Ia or Ib, glycerol monocaprylocaprate, gelatin, glycerin, titanium dioxide, and iron oxide comprises iron oxide (yellow). In some embodiments, the hard gelatin or soft capsule comprises about 65.9 w/w % of glycerol monocaprylocaprate, about 3.4 w/w % of a compound of Formula Ia or Ib, about 19.6 w/w % of gelatin, about 10.8 w/w % of glycerin, about 0.2 w/w % of titanium dioxide, and about 0.02 w/w % of iron oxide (yellow). In some embodiments, the hard gelatin or soft capsule comprises about 65.94 w/w % of glycerol monocaprylocaprate, about 3.42 w/w % of a compound of Formula Ia or Ib, about 19.60 w/w % of gelatin, about 10.80 w/w % of glycerin, about 0.22 w/w % of titanium dioxide, and about 0.02 w/w % of iron oxide (yellow).

In some embodiments, the hard gelatin or soft gelatin capsule comprises a sodium salt of the compound of Formula Ia or Ib and glycerol monocaprylocaprate. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml to about 500 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml to about 400 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 50 mg/ml to about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 75 mg/ml to about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 10 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 20 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 30 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 40 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 50 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 75 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 100 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 125 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 150 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 175 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 200 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 225 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 250 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 275 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 300 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 325 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 350 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 375 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 400 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 425 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 450 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 475 mg/ml. In some embodiments, the concentration of the compound of Formula Ia or Ib in the hard gelatin or soft gelatin capsule comprising a sodium salt of the compound of Formula Ia or Ib and glycerol monocaprylocaprate is about 500 mg/ml.

In some embodiments, the amount of glycerol monocaprylocaprate in the hard gelatin or soft gelatin capsule comprising a sodium salt of the compound of Formula Ia or Ib and glycerol monocaprylocaprate is about 30 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 50 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 60 w/w % to about 99 w/w %. In some embodiments, the amount of glycerol monocaprylocaprate in the hard gelatin or soft gelatin capsule comprising a sodium salt of the compound of Formula Ia or Ib and glycerol monocaprylocaprate is about 75 w/w % to about 98 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.09 w/w % to about 94.85 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.1 w/w % to about 94.9 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.09 w/w %. In some embodiments, the amount of glycerol monocaprylocaprate in the hard gelatin or soft gelatin capsule comprising a sodium salt of the compound of Formula Ia or Ib and glycerol monocaprylocaprate is about 80.1 w/w %. In some embodiments, the amount of glycerol monocaprylocaprate in the hard gelatin or soft gelatin capsule comprising a sodium salt of the compound of Formula Ia or Ib and glycerol monocaprylocaprate is about 94.85 w/w %. In some embodiments, the amount of glycerol monocaprylocaprate in the hard gelatin or soft gelatin capsule comprising a sodium salt of the compound of Formula Ia or Ib and glycerol monocaprylocaprate is about 94.9 w/w %.

In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 1 w/w % to about 40 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 1 w/w % to about 35 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 2 w/w % to about 30 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 3 w/w % to about 28 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 5.15 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 5.2 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 19.9 w/w %. In some embodiments, the amount of the sodium salt of the compound of Formula Ia or Ib in the hard gelatin or soft gelatin capsule comprising a sodium salt of the compound of Formula Ia or Ib and glycerol monocaprylocaprate is about 19.91 w/w %.

In some embodiments, the hard gelatin or soft gelatin capsule comprising a sodium salt of the compound of Formula Ia or Ib and glycerol monocaprylocaprate further comprises a capsule shell. In some embodiments, the capsule shell comprises one or more pharmaceutically acceptable excipients. In some embodiments, the one or more pharmaceutically acceptable excipients of the capsule shell is selected from the group consisting of a gelatin shell, a plasticizer, an opacifier, and a colorant. In some embodiments, the hard gelatin or soft gelatin capsule comprising a sodium salt of the compound of Formula Ia or Ib and glycerol monocaprylocaprate further comprises one or more of a pharmaceutically acceptable excipient selected from the group consisting of gelatin, glycerin, titanium dioxide, and iron oxide. In some embodiments, the hard gelatin or soft gelatin capsule comprising a sodium salt of the compound of Formula Ia or Ib and glycerol monocaprylocaprate further comprises gelatin. In some embodiments, the hard gelatin or soft gelatin capsule comprising a sodium salt of the compound of Formula Ia or Ib and glycerol monocaprylocaprate further comprises glycerin. In some embodiments, the hard gelatin or soft gelatin capsule comprising a sodium salt of the compound of Formula Ia or Ib and glycerol monocaprylocaprate further comprises titanium dioxide. In some embodiments, the hard gelatin or soft gelatin capsule comprising a sodium salt of the compound of Formula Ia or Ib and glycerol monocaprylocaprate further comprises iron oxide. In some embodiments, the hard gelatin or soft gelatin capsule comprising a sodium salt of the compound of Formula Ia or Ib and glycerol monocaprylocaprate further comprises gelatin and glycerin. In some embodiments, the hard gelatin or soft gelatin capsule comprising a sodium salt of the compound of Formula Ia or Ib and glycerol monocaprylocaprate further comprises gelatin, glycerin, and titanium dioxide. In some embodiments, the hard gelatin or soft gelatin capsule comprising a sodium salt of the compound of Formula Ia or Ib and glycerol monocaprylocaprate further comprises gelatin, glycerin, titanium dioxide, and iron oxide. In some embodiments, the iron oxide of the hard gelatin or soft gelatin capsule comprising a sodium salt of the compound of Formula Ia or Ib, glycerol monocaprylocaprate, gelatin, glycerin, titanium dioxide, and iron oxide comprises iron oxide (yellow).

In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 10 w/w % to about 40 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 10 w/w % to about 30 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 15 w/w % to about 25 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 18 w/w % to about 22 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 19.6 w/w %. In some embodiments, the amount of gelatin in the hard gelatin or soft gelatin capsule comprising a sodium salt of the compound of Formula Ia or Ib, glycerol monocaprylocaprate, gelatin, glycerin, titanium dioxide, and iron oxide is about 19.60 w/w %.

In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 3 w/w % to about 25 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 5 w/w % to about 20 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 5 w/w % to about 15 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 8 w/w % to about 12 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 10.8 w/w %. In some embodiments, the amount of glycerin in the hard gelatin or soft gelatin capsule comprising a sodium salt of the compound of Formula Ia or Ib, glycerol monocaprylocaprate, gelatin, glycerin, titanium dioxide, and iron oxide is about 10.80 w/w %.

In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 2 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.05 w/w % to about 1.5 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.1 w/w % to about 1.0 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.1 w/w % to about 0.5 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.2 w/w %. In some embodiments, the amount of titanium dioxide in the hard gelatin or soft gelatin capsule comprising a sodium salt of the compound of Formula Ia or Ib, glycerol monocaprylocaprate, gelatin, glycerin, titanium dioxide, and iron oxide is about 0.22 w/w %.

In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 1 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.5 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.15 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.01 w/w % to about 0.1 w/w %. In some embodiments, the amount of iron oxide in the hard gelatin or soft gelatin capsule comprising a sodium salt of the compound of Formula Ia or Ib, glycerol monocaprylocaprate, gelatin, glycerin, titanium dioxide, and iron oxide is about 0.02 w/w %.

In some embodiments, the hard gelatin or soft capsule disclosed herein comprises about 30 w/w % to about 99 w/w % of glycerol monocaprylocaprate and about 1 w/w % to about 40 w/w % of a sodium salt of the compound of Formula Ia or Ib. In some embodiments, the hard gelatin or soft capsule comprises about 50 w/w % to about 99 w/w % of glycerol monocaprylocaprate and about 1 w/w % to about 35 w/w % of a sodium salt of the compound of Formula Ia or Ib. In some embodiments, the hard gelatin or soft capsule comprises about 60 w/w % to about 99 w/w % of glycerol monocaprylocaprate and about 2 w/w % to about 30 w/w % of a sodium salt of the compound of Formula Ia or Ib. In some embodiments, the hard gelatin or soft capsule comprises about 75 w/w % to about 98 w/w % of glycerol monocaprylocaprate and about 3 w/w % to about 28 w/w % of a sodium salt of the compound of Formula Ia or Ib. In some embodiments, the hard gelatin or soft capsule comprises about 80.09 w/w % to about 94.85 w/w % of glycerol monocaprylocaprate and about 5.15 w/w % to about 19.91 w/w % of a sodium salt of the compound of Formula Ia or Ib. In some embodiments, the hard gelatin or soft capsule comprises about 80.1 w/w % to about 94.9 w/w % of glycerol monocaprylocaprate and about 5.2 w/w % to about 19.9 w/w % of a sodium salt of the compound of Formula Ia or Ib. In some embodiments, the hard gelatin or soft capsule comprises about 94.85 w/w % of glycerol monocaprylocaprate and about 5.15 w/w % of a sodium salt of the compound of Formula Ia or Ib. In some embodiments, the hard gelatin or soft capsule comprises about 94.9 w/w % of glycerol monocaprylocaprate and about 5.2 w/w % of a sodium salt of the compound of Formula Ia or Ib.

In some embodiments, the hard gelatin or soft capsule comprises about 80.09 w/w % of glycerol monocaprylocaprate and about 19.91 w/w % of a sodium salt of the compound of Formula Ia or Ib. In some embodiments, the hard gelatin or soft capsule comprises about 80.1 w/w % of glycerol monocaprylocaprate and about 19.9 w/w % of a sodium salt of the compound of Formula Ia or Ib.

In some embodiments, the iron oxide in the hard gelatin or soft gelatin capsule comprising a sodium salt of the compound of Formula Ia or Ib, glycerol monocaprylocaprate, gelatin, glycerin, titanium dioxide, and iron oxide comprises iron oxide (yellow).

In some embodiments, the oral formulation of a compound of Formula Ia or Ib, or a pharmaceutically acceptable salt thereof, is a tablet. In some embodiments, the tablet is prepared from a spray-dried dispersion of a compound of Formula Ia or Ib, or a pharmaceutically acceptable salt thereof. In some embodiments, the amount of a compound of Formula Ia or Ib, or a pharmaceutically acceptable salt thereof, in the tablet is about 5 mg to about 500 mg. In some embodiments, the amount of a compound of Formula Ia or Ib, or a pharmaceutically acceptable salt thereof, in the tablet is about 25 mg to about 500 mg. In some embodiments, the amount of a compound of Formula Ia or Ib, or a pharmaceutically acceptable salt thereof, in the tablet is about 25 mg to about 400 mg. In some embodiments, the amount of a compound of Formula Ia or Ib, or a pharmaceutically acceptable salt thereof, in the tablet is about 25 mg to about 300 mg. In some embodiments, the amount of a compound of Formula Ia or Ib, or a pharmaceutically acceptable salt thereof, in the tablet is about 50 mg to about 500 mg.

In some embodiments, the amount of a compound of Formula Ia or Ib, or a pharmaceutically acceptable salt thereof, in the tablet is about 75 mg to about 500 mg. In some embodiments, the amount of a compound of Formula Ia or Ib, or a pharmaceutically acceptable salt thereof, in the tablet is about 50 mg to about 400 mg. In some embodiments, the amount of a compound of Formula Ia or Ib, or a pharmaceutically acceptable salt thereof, in the tablet is about 50 mg to about 300 mg. In some embodiments, the amount of a compound of Formula Ia or Ib, or a pharmaceutically acceptable salt thereof, in the tablet is about 75 mg to about 400 mg. In some embodiments, the amount of a compound of Formula Ia or Ib, or a pharmaceutically acceptable salt thereof, in the tablet is about 75 mg to about 300 mg. In some embodiments, the amount of a compound of Formula Ia or Ib, or a pharmaceutically acceptable salt thereof, in the tablet is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg. In some embodiments, the amount of a compound of Formula Ia or Ib, or a pharmaceutically acceptable salt thereof, in the tablet is about 5 mg. In some embodiments, the amount of a compound of Formula Ia or Ib, or a pharmaceutically acceptable salt thereof, in the tablet is about 10 mg. In some embodiments, the amount of a compound of Formula Ia or Ib, or a pharmaceutically acceptable salt thereof, in the tablet is about 20 mg. In some embodiments, the amount of a compound of Formula Ia or Ib, or a pharmaceutically acceptable salt thereof, in the tablet is about 25 mg. In some embodiments, the amount of a compound of Formula Ia or Ib, or a pharmaceutically acceptable salt thereof, in the tablet is about 30 mg. In some embodiments, the amount of a compound of Formula Ia or Ib, or a pharmaceutically acceptable salt thereof, in the tablet is about 40 mg. In some embodiments, the amount of a compound of Formula Ia or Ib, or a pharmaceutically acceptable salt thereof, in the tablet is about 50 mg. In some embodiments, the amount of a compound of Formula Ia or Ib, or a pharmaceutically acceptable salt thereof, in the tablet is about 75 mg. In some embodiments, the amount of a compound of Formula Ia or Ib, or a pharmaceutically acceptable salt thereof, in the tablet is about 100 mg. In some embodiments, the amount of a compound of Formula Ia or Ib, or a pharmaceutically acceptable salt thereof, in the tablet is about 125 mg. In some embodiments, the amount of a compound of Formula Ia or Ib, or a pharmaceutically acceptable salt thereof, in the tablet is about 150 mg. In some embodiments, the amount of a compound of Formula Ia or Ib, or a pharmaceutically acceptable salt thereof, in the tablet is about 175 mg. In some embodiments, the amount of a compound of Formula Ia or Ib, or a pharmaceutically acceptable salt thereof, in the tablet is about 200 mg. In some embodiments, the amount of a compound of Formula Ia or Ib, or a pharmaceutically acceptable salt thereof, in the tablet is about 225 mg. In some embodiments, the amount of a compound of Formula Ia or Ib, or a pharmaceutically acceptable salt thereof, in the tablet is about 250 mg. In some embodiments, the amount of a compound of Formula Ia or Ib, or a pharmaceutically acceptable salt thereof, in the tablet is about 275 mg. In some embodiments, the amount of a compound of Formula Ia or Ib, or a pharmaceutically acceptable salt thereof, in the tablet is about 300 mg.

In some embodiments, the amount of a compound of Formula Ia or Ib, or a pharmaceutically acceptable salt thereof, in the tablet is about 325 mg. In some embodiments, the amount of a compound of Formula Ia or Ib, or a pharmaceutically acceptable salt thereof, in the tablet is about 350 mg. In some embodiments, the amount of a compound of Formula Ia or Ib, or a pharmaceutically acceptable salt thereof, in the tablet is about 375 mg. In some embodiments, the amount of a compound of Formula Ia or Ib, or a pharmaceutically acceptable salt thereof, in the tablet is about 400 mg. In some embodiments, the amount of a compound of Formula Ia or Ib, or a pharmaceutically acceptable salt thereof, in the tablet is about 425 mg. In some embodiments, the amount of a compound of Formula Ia or Ib, or a pharmaceutically acceptable salt thereof, in the tablet is about 450 mg. In some embodiments, the amount of a compound of Formula Ia or Ib, or a pharmaceutically acceptable salt thereof, in the tablet is about 475 mg. In some embodiments, the amount of a compound of Formula Ia or Ib, or a pharmaceutically acceptable salt thereof, in the tablet is about 500 mg.

In some embodiments, the tablet herein comprises a compound of Formula Ia or Ib, or a pharmaceutically acceptable salt thereof, and one or more pharmaceutically acceptable excipients. In some embodiments, the tablet comprises a compound of Formula Ia or a pharmaceutically acceptable salt thereof, and one or more pharmaceutically acceptable excipients. In some embodiments, the tablet comprises a compound of Formula Ia and one or more pharmaceutically acceptable excipients. In some embodiments, the tablet comprises a sodium salt of the compound of Formula Ia and one or more pharmaceutically acceptable excipients. In some embodiments, the tablet comprises a compound of Formula Ib or a pharmaceutically acceptable salt thereof, and one or more pharmaceutically acceptable excipients. In some embodiments, the tablet comprises a compound of Formula Ib and one or more pharmaceutically acceptable excipients. In some embodiments, the tablet comprises a sodium salt of the compound of Formula Ib and one or more pharmaceutically acceptable excipients.

The pharmaceutically acceptable excipients of the tablets disclosed herein should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6th edition 2009. As used herein the term “excipients” is intended to refer to inter alia basifying agents, solubilisers, glidants, fillers, binders, lubricant, diluents, preservatives, surface active agents, dispersing agents and the like. The term also includes agents such as sweetening agents, flavoring agents, coloring agents and preserving agents. Such components will generally be present in admixture within the tablet.

Examples of solubilisers include, but are not limited to, surfactants (including both ionic and non-ionic surfactants) such as sodium lauryl sulphate, cetyltrimethylammonium bromide, polysorbates (such as polysorbate 20 or 80), poloxamers (such as poloxamer 188 or 207), and macrogols. Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc.

Examples of disintegrants include, but are not limited to, starches, celluloses, cross-linked PVP, sodium starch glycolate, croscarmellose sodium, and the like. Examples of fillers (also known as bulking agents or diluents) include, but are not limited to, starches, maltodextrins, polyols (such as lactose), and celluloses. Examples of binders include, but are not limited to, cross-linked PVP, HPMC, microcrystalline cellulose, sucrose, starches, and the like.

In some embodiments, the tablets disclosed herein comprise a compound of Formula Ia or Ib, or a pharmaceutically acceptable salt thereof, and one or more pharmaceutically acceptable excipients selected from the group consisting of a matrix former, a surfactant, a filler, a disintegrant, and a lubricant. In some embodiments, the tablet comprises about 1 w/w % to about 10 w/w % of a matrix former. In some embodiments, the matrix former comprises copovidone. In some embodiments, the tablet comprises about 0.01 w/w % to about 10 w/w % of a surfactant. In some embodiments, the surfactant comprises poloxamer 407. In some embodiments, the tablet comprises about 25-85 w/w % of one or more fillers. In some embodiments, the one or more fillers comprises microcrystalline cellulose and/or mannitol. In some embodiments, the tablet comprises about 1 w/w % to about 30 w/w % of a disintegrant. In some embodiments, the disintegrant comprises croscarmellose sodium. In some embodiments, the tablet comprises about 0.01 w/w % to about 10 w/w % of a lubricant. In some embodiments, the lubricant comprises magnesium stearate.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (IPMC), methylcellulose, hydroxyethyl cellulose (HEC), hydroxypropyl cellulose (HIPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight.

In some embodiments, the tablet disclosed herein comprises a compound of Formula Ia or Ib or a pharmaceutically acceptable salt thereof, and one or more pharmaceutically acceptable excipients. In some embodiments, the one or more pharmaceutically acceptable excipients is selected from the group consisting of copovidone, poloxamer 407, microcrystalline cellulose, mannitol, croscarmellose sodium, and magnesium stearate. In some embodiments, the one or more pharmaceutically acceptable excipient comprises copovidone. In some embodiments, the one or more pharmaceutically acceptable excipient comprises poloxamer 407. In some embodiments, the one or more pharmaceutically acceptable excipient comprises microcrystalline cellulose. In some embodiments, the one or more pharmaceutically acceptable excipient comprises mannitol. In some embodiments, the one or more pharmaceutically acceptable excipient comprises croscarmellose sodium. In some embodiments, the one or more pharmaceutically acceptable excipient comprises magnesium stearate. In some embodiments, the one or more pharmaceutically acceptable excipient comprises copovidone and poloxamer 407. In some embodiments, the one or more pharmaceutically acceptable excipient comprises copovidone, poloxamer 407, and microcrystalline cellulose. In some embodiments, the one or more pharmaceutically acceptable excipient comprises copovidone, poloxamer 407, microcrystalline cellulose, and mannitol. In some embodiments, the one or more pharmaceutically acceptable excipient comprises copovidone, poloxamer 407, microcrystalline cellulose, mannitol, and croscarmellose sodium. In some embodiments, the one or more pharmaceutically acceptable excipient comprises copovidone, poloxamer 407, microcrystalline cellulose, mannitol, croscarmellose sodium, and magnesium stearate.

In some embodiments, the tablet disclosed herein comprises a compound of Formula Ia or Ib or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ib, or a pharmaceutically acceptable salt thereof, copovidone, poloxamer 407, microcrystalline cellulose, mannitol, croscarmellose sodium, and magnesium stearate. In some embodiments, the tablet comprises a compound of Formula Ia, copovidone, poloxamer 407, microcrystalline cellulose, mannitol, croscarmellose sodium, and magnesium stearate. In some embodiments, the tablet comprises a sodium salt of the compound of Formula Ia, copovidone, poloxamer 407, microcrystalline cellulose, mannitol, croscarmellose sodium, and magnesium stearate. In some embodiments, the tablet comprises a compound of Formula Ib, copovidone, poloxamer 407, microcrystalline cellulose, mannitol, croscarmellose sodium, and magnesium stearate. In some embodiments, the tablet comprises a sodium salt of the compound of Formula Ib, copovidone, poloxamer 407, microcrystalline cellulose, mannitol, croscarmellose sodium, and magnesium stearate.

In some embodiments, the tablet disclosed herein comprises a sodium salt of the compound of Formula Ia or Ib, copovidone, poloxamer 407, microcrystalline cellulose, mannitol, croscarmellose sodium, and magnesium stearate. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 500 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg to about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 25 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 30 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 40 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 50 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 75 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 100 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 125 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 150 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 175 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 200 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 225 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 250 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 275 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 300 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 325 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 350 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 375 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 400 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 425 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 450 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 475 mg. In some embodiments, the amount of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 500 mg.

In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 15 w/w % to about 25 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.46 w/w %. In some embodiments, the amount of the sodium salt of the compound of Formula Ia or Ib in the tablet comprising a sodium salt of the compound of Formula Ia or Ib, copovidone, poloxamer 407, microcrystalline cellulose, mannitol, croscarmellose sodium, and magnesium stearate is about 20.5 w/w %.

In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 1 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 2 w/w % to about 10 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 8 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 3 w/w % to about 6 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.88 w/w %. In some embodiments, the amount of copovidone in the tablet comprising a sodium salt of the compound of Formula Ia or Ib, copovidone, poloxamer 407, microcrystalline cellulose, mannitol, croscarmellose sodium, and magnesium stearate is about 4.9 w/w %.

In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 3.0 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.3 w/w %. In some embodiments, the amount of poloxamer 407 in the tablet comprising a sodium salt of the compound of Formula Ia or Ib, copovidone, poloxamer 407, microcrystalline cellulose, mannitol, croscarmellose sodium, and magnesium stearate is about 1.33 w/w %.

In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 5 w/w % to about 45 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 10 w/w % to about 40 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 15 w/w % to about 35 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 18 w/w % to about 30 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.28 w/w %. In some embodiments, the amount of microcrystalline cellulose in the tablet comprising a sodium salt of the compound of Formula Ia or Ib, copovidone, poloxamer 407, microcrystalline cellulose, mannitol, croscarmellose sodium, and magnesium stearate is about 21.3 w/w %.

In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 15 w/w % to about 70 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 20 w/w % to about 60 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 30 w/w % to about 55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 40 w/w % to about 50 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.55 w/w %. In some embodiments, the amount of mannitol in the tablet comprising a sodium salt of the compound of Formula Ia or Ib, copovidone, poloxamer 407, microcrystalline cellulose, mannitol, croscarmellose sodium, and magnesium stearate is about 42.6 w/w %.

In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 30 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 1 w/w % to about 20 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 4 w/w % to about 16 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 6 w/w % to about 10 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.0 w/w %. In some embodiments, the amount of croscarmellose sodium in the tablet comprising a sodium salt of the compound of Formula Ia or Ib, copovidone, poloxamer 407, microcrystalline cellulose, mannitol, croscarmellose sodium, and magnesium stearate is about 8.00 w/w %.

In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.01 w/w % to about 10 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.05 w/w % to about 8 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 0.5 w/w % to about 4 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.0 w/w % to about 3.0 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.5 w/w %. In some embodiments, the amount of magnesium stearate in the tablet comprising a sodium salt of the compound of Formula Ia or Ib, copovidone, poloxamer 407, microcrystalline cellulose, mannitol, croscarmellose sodium, and magnesium stearate is about 1.50 w/w %.

In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, about 0.01 w/w % to about 10 w/w % of magnesium stearate, and one or more pharmaceutically acceptable excipients. In some embodiments, the tablet disclosed herein comprises about 5 w/w % to about 45 w/w % of a sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate. In some embodiments, the tablet comprises about 10 w/w % to about 40 w/w % of a sodium salt of the compound of Formula Ia or Ib, about 2 w/w % to about 10 w/w % of copovidone, about 0.05 w/w % to about 8 w/w % of poloxamer 407, about 10 w/w % to about 40 w/w % of microcrystalline cellulose, about 20 w/w % to about 60 w/w % of mannitol, about 1 w/w % to about 20 w/w % of croscarmellose sodium, and about 0.05 w/w % to about 8 w/w % of magnesium stearate. In some embodiments, the tablet comprises about 15 w/w % to about 35 w/w % of a sodium salt of the compound of Formula Ia or Ib, about 3 w/w % to about 8 w/w % of copovidone, about 0.5 w/w % to about 4 w/w % of poloxamer 407, about 15 w/w % to about 35 w/w % of microcrystalline cellulose, about 30 w/w % to about 55 w/w % of mannitol, about 4 w/w % to about 16 w/w % of croscarmellose sodium, and about 0.5 w/w % to about 4 w/w % of magnesium stearate. In some embodiments, the tablet comprises about 15 w/w % to about 25 w/w % of a sodium salt of the compound of Formula Ia or Ib, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % of magnesium stearate.

In some embodiments, the tablet comprises about 20.5 w/w % of a sodium salt of the compound of Formula Ia or Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ia or Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate. In some embodiments, the tablet comprises about 20.5 w/w % of a sodium salt of the compound of Formula Ib, about 4.9 w/w % of copovidone, about 1.3 w/w % of poloxamer 407, about 21.3 w/w % of microcrystalline cellulose, about 42.6 w/w % of mannitol, about 8.0 w/w % of croscarmellose sodium, and about 1.5 w/w % of magnesium stearate. In some embodiments, the tablet comprises about 20.46 w/w % of a sodium salt of the compound of Formula Ib, about 4.88 w/w % of copovidone, about 1.33 w/w % of poloxamer 407, about 21.28 w/w % of microcrystalline cellulose, about 42.55 w/w % of mannitol, about 8.00 w/w % of croscarmellose sodium, and about 1.50 w/w % of magnesium stearate.

In some embodiments, the tablet disclosed herein further comprises an outer film coat. In some embodiments, the tablet comprising a sodium salt of the compound of Formula Ia or Ib, copovidone, poloxamer 407, microcrystalline cellulose, mannitol, croscarmellose sodium, and magnesium stearate further comprises an outer film coat. In some embodiments, the outer film coat provides from about 1% to about 8% weight gain based on the uncoated tablet. In some embodiments, the outer film coat provides from about 2% to about 6% weight gain based on the uncoated tablet. In some embodiments, the outer film coat provides from about 2% to about 4% weight gain based on the uncoated tablet. In some embodiments, the outer film coat provides from about 4% to about 6% weight gain based on the uncoated tablet. In some embodiments, the outer film coat provides about 1%, about 2%, about 3%, about 4%, about 5%, about 6%, about 7%, or about 8% weight gain based on the uncoated tablet. In some embodiments, the outer film coat provides about 1% weight gain based on the uncoated tablet. In some embodiments, the outer film coat provides about 2% weight gain based on the uncoated tablet. In some embodiments, the outer film coat provides about 3% weight gain based on the uncoated tablet. In some embodiments, the outer film coat provides about 4% weight gain based on the uncoated tablet. In some embodiments, the outer film coat provides about 5% weight gain based on the uncoated tablet. In some embodiments, the outer film coat provides about 6% weight gain based on the uncoated tablet. In some embodiments, the outer film coat provides about 7% weight gain based on the uncoated tablet. In some embodiments, the outer film coat provides about 8% weight gain based on the uncoated tablet. In some embodiments, the outer film coat comprises Opadry® II. In some embodiments, the outer film coat comprises Opadry® II White. In some embodiments, the outer film coat comprises Opadry® II White 85F18422.

In some embodiments, the outer film coat comprises Opadry® II Green. In some embodiments, the outer film coat comprises Opadry® II Green 85F110187. In some embodiments, the outer film coat comprises Opadry® II Green 85F110186.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 3% weight gain from Opadry® II White 85F18422, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 3.0% weight gain from Opadry® II White 85F18422, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110187, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110187, wherein the weight gain is based on the uncoated tablet.

In some embodiments, the tablet comprises about 20.5 w/w % of a sodium salt of a compound of Formula Ia or Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ia or Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet. In some embodiments, the tablet comprises about 20.5 w/w % of a sodium salt of a compound of Formula Ib, about 4.9 w/w % copovidone, about 1.3 w/w % poloxamer 407, about 21.3 w/w % of microcrystalline cellulose, about 42.6 w/w % of mannitol, about 8.0 w/w % of croscarmellose sodium, about 1.5 w/w % of magnesium stearate, and about 4% weight gain from Opadry® II Green 85F110186, wherein the weight gain is based on the uncoated tablet. In some embodiments, the tablet comprises about 20.46 w/w % of a sodium salt of a compound of Formula Ib, about 4.88 w/w % copovidone, about 1.33 w/w % poloxamer 407, about 21.28 w/w % of microcrystalline cellulose, about 42.55 w/w % of mannitol, about 8.00 w/w % of croscarmellose sodium, about 1.50 w/w % of magnesium stearate, and about 4.0% weight gain from Opadry® II Green 85F110186, wherein the weight gain is based on the uncoated tablet.

The pharmaceutical compositions disclosed herein can be also prepared by other methodologies known in the pharmaceutical art. For example, in certain embodiments, a pharmaceutical composition intended to be administered by injection can prepared by combining the compound of Formula Ia or Ib, or a pharmaceutically acceptable salt thereof, with sterile, distilled water so as to form a solution. In some embodiments, a surfactant is added to facilitate the formation of a homogeneous solution or suspension. Surfactants are compounds that non-covalently interact with the compound of Formula Ia or Ib, or a pharmaceutically acceptable salt thereof, so as to facilitate dissolution or homogeneous suspension of the compound in the aqueous delivery system.

The terms “effective amount” or “therapeutically effective amount” refer to an amount of the compound of Formula Ia or Ib, or other anti-HIV agent, or a pharmaceutically acceptable salt thereof, which when administered to a patient in need thereof, is sufficient to effect preventing an HIV infection or reducing the risk of contracting HIV infection, as described herein. Such an amount would be sufficient to elicit the biological or medical response of a tissue system, or patient that is sought by a researcher or clinician. The amount of the compound of Formula Ia or Ib or other anti-HIV agent which constitutes a therapeutically effective amount will vary depending on such factors as the compound, salt, or composition used for administration, the time of administration, the route of administration, the rate of excretion of the compound, the duration of the treatment, the type of disease-state or disorder being treated and its severity, drugs used in combination with or coincidentally with the compound of Formula Ia or Ib, and the age, body weight, general health, sex and diet of the patient. Such a therapeutically effective amount can be determined routinely by one of ordinary skill in the art having regard to their own knowledge, the state of the art, and this disclosure.

In some embodiments of the methods provided herein, each oral administration is administered as a tablet comprising the sodium salt of the compound of Formula Ia or Ib.

In some embodiments of the methods provided herein, each tablet is prepared from a spray-dried dispersion technology.

In some embodiments of the methods provided herein, each tablet comprises about 5 w/w % to about 45 w/w % of the sodium salt of the compound of Formula Ia or Ib, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate, and one or more pharmaceutically acceptable excipients.

In some embodiments of the methods provided herein, each tablet comprises about 15 w/w % to about 25 w/w % of the sodium salt of the compound of Formula Ia or Ib, about 3 w/w % to about 6 w/w % of copovidone, about 0.5 w/w % to about 3.0 w/w % of poloxamer 407, about 18 w/w % to about 30 w/w % of microcrystalline cellulose, about 40 w/w % to about 50 w/w % of mannitol, about 6 w/w % to about 10 w/w % of croscarmellose sodium, and about 1.0 w/w % to about 3.0 w/w % magnesium stearate.

In some embodiments of the methods provided herein, each tablet comprises about 300 mg of the compound of Formula Ia, or a pharmaceutically acceptable salt thereof.

In some embodiments of the methods provided herein, each tablet comprises about 306.8 mg of the sodium salt of the compound of Formula Ia or Ib.

In some embodiments of the methods provided herein, each tablet further comprises an outer film coat.

In some embodiments of the methods provided herein, the outer film coat provides from about 1% to about 8% weight gain based on the uncoated tablet. In some embodiments of the methods provided herein, the outer film coat provides from about 2% to about 5% weight gain based on the uncoated tablet.

In some embodiments of the methods provided herein, wherein the outer film coat provides about 2% weight gain based on the uncoated tablet. In some embodiments of the methods provided herein, wherein the outer film coat provides about 3% weight gain based on the uncoated tablet. In some embodiments of the methods provided herein, wherein the outer film coat provides about 4% weight gain based on the uncoated tablet. In some embodiments of the methods provided herein, wherein the outer film coat provides about 5% weight gain based on the uncoated tablet.

### Combination Therapies

Patients being treated by administration of the compound of Formula Ia or Ib, or a pharmaceutically acceptable salt thereof, often exhibit diseases or conditions that benefit from treatment with other therapeutic agents, including agents that are therapeutic for Retroviridae infections, including an HIV infection. In some embodiments, the other therapeutic agent is an agent that is therapeutic for an HIV infection. Thus, one aspect of the disclosure is a method of treating an HIV infection comprising administering a compound of Formula Ia or Ib, or a pharmaceutically acceptable salt thereof, in combination with one or more compounds useful for the treatment of an HIV infection to a patient in need thereof.

In some embodiments, a compound of Formula Ia or Ib, or a pharmaceutically acceptable salt thereof, is combined with one, two, three, four or more additional therapeutic agents. In some embodiments, a compound of Formula Ia or Ib, or a pharmaceutically acceptable salt thereof, is combined with two additional therapeutic agents. In some embodiments, a compound of Formula Ia or Ib, or a pharmaceutically acceptable salt thereof, is combined with three additional therapeutic agents. In some embodiments, a compound of Formula Ia or Ib, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of Formula Ia or Ib, or a pharmaceutically acceptable salt thereof, is combined with one or more additional therapeutic agents as described herein, the components of the composition are administered as a simultaneous or sequential regimen.

In some embodiments, a compound of Formula Ia or Ib, or a pharmaceutically acceptable salt thereof, and the additional therapeutic agents are administered simultaneously.

In some embodiments, a compound of Formula Ia or Ib, or a pharmaceutically acceptable salt thereof, and the additional therapeutic agents are administered sequentially. When administered sequentially, the combination may be administered in two or more administrations.

In some embodiments, a compound of Formula Ia or Ib, or a pharmaceutically acceptable salt thereof, is combined with one or more additional therapeutic agents in a unitary dosage form for simultaneous administration to a patient.

In some embodiments, a compound of Formula Ia or Ib, or a pharmaceutically acceptable salt thereof, is co-administered with one or more additional therapeutic agents.

Co-administration includes administration of unit dosages of the compounds provided herein, or pharmaceutically acceptable salts thereof, before or after administration of unit dosages of one or more additional therapeutic agents. The compound of Formula Ia or Ib, or pharmaceutically acceptable salts thereof, may be administered within seconds, minutes, or hours of the administration of one or more additional therapeutic agents. For example, in some embodiments, a unit dose of a compound of Formula Ia or Ib, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of Formula Ia or Ib, or a pharmaceutically acceptable salt thereof, within seconds or minutes. In some embodiments, a unit dose of a compound of Formula Ia or Ib, or a pharmaceutically acceptable salt thereof, is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of a compound of Formula Ia or Ib, or a pharmaceutically acceptable salt thereof.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents any are chosen from dolutegravir, cabotegravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), elsulfavirine (long acting injectable nanosuspension, HIV infection), and elsulfavirine (VM-1500). Additional non-limiting examples of non-nucleoside or non-nucleotide inhibitors of reverse transcriptase include the compounds disclosed in U.S. Pat. No. 10,548,898.

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017. Additional non-limiting examples of HIV integrase inhibitors include the compounds disclosed in U.S. Pat. No. 11,084,832.

Examples of HIV non-catalytic site, or allosteric, integrase inhibitors (NCINI) include, but are not limited to, CX-05045, CX-05168, and CX-14442.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HIBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

Additional examples of HIV capsid inhibitors include, but are not limited to, those described in U.S. Patent Application Publication Nos. US2014221356 and US2016016973.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KTR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JUL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMNC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, 5-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-1 and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4 Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):D1 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):D1 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-Bi1, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound of Formula Ia or Ib, or a pharmaceutically acceptable salt thereof, may be combined with one, two, three, or four additional therapeutic agents in any dosage amount of the compound of Formula Ia or Ib, or a pharmaceutically acceptable salt thereof.

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, doravirine, raltegravir, and long acting dolutegravir.

In some embodiments, the methods provided herein further comprise administering one, two, three, or four additional therapeutic agents to the patient.

In some embodiments of the methods provided herein, the additional therapeutic agents are selected from the group consisting of combination drugs for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof.

In some embodiments of the methods provided herein, the additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, or any combinations thereof.

In some embodiments of the methods provided herein, the additional therapeutic agents are selected from the group consisting of bictegravir or a pharmaceutically acceptable salt thereof, abacavir sulfate, tenofovir, tenofovir disoproxil, tenofovir disoproxil fumarate, tenofovir disoproxil hemifumarate, tenofovir alafenamide, and tenofovir alafenamide hemifumarate.

In some embodiments of the methods provided herein, the additional therapeutic agents are selected from the group consisting of bictegravir or a pharmaceutically acceptable salt thereof, tenofovir alafenamide, tenofovir alafenamide fumarate, and tenofovir alafenamide hemifumarate.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Pharmacokinetics Modeling Simulations of Lenacapavir Oral Dosing Regimen

The pharmacokinetics (PK) of lenacapavir (LEN) following single-dose administration of oral LEN 50-1800 mg has been characterized; oral LEN exposure increased less than dose proportionally over the evaluated dose range, with maximal plasma concentrations between 4 and 8 hours postdose, and a half-life of 10-13 days. Mean trough concentration of 15.5 ng/mL, which is inhibitory quotient 4 (IQ4; ie, 4-fold greater than the in vitro protein-adjusted 95% effective concentration derived from MT-4 cells) has been associated with high rates of virologic suppression in Phase 2/3 clinical studies.

The purpose of this analysis was to identify LEN dosing regimens that can be used, for example, in combinations with other antiretroviral agents by simulating various weekly dosing regimens that would rapidly achieve and maintain LEN concentrations above inhibitory quotient 4 (IQ4).

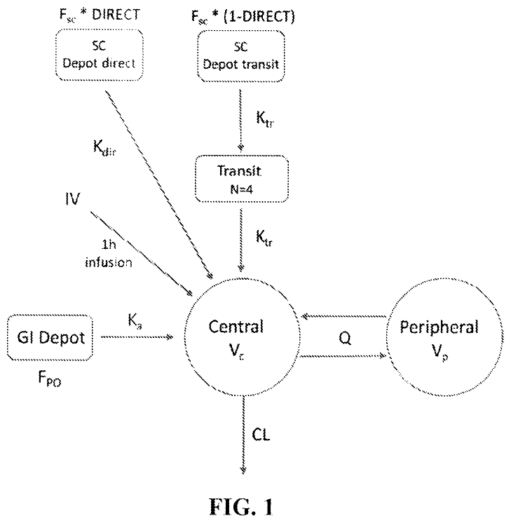

PK data were pooled from several clinical studies in healthy participants and people with HIV-1 who received intravenous (IV)/oral/SC LEN and a 2-compartment LEN population pharmacokinetic (PopPK) model with first-order absorption and linear elimination was built to simulate various weekly dosing regimens (loading+maintenance doses) that can achieve efficacious LEN concentrations rapidly and be maintained through the dosing interval. Various scenarios including 1-3 weeks of missed oral dosing were simulated to evaluate the forgiveness window; these simulations were performed with the PopPK model incorporating variability and covariate effects. A summary of the model is shown in FIG. 1. The model pharmacokinetic parameters for lenacapavir are provided in Table 1 where CL=clearance; Fₚₒ=oral bioavailability; F_{sc}=SC bioavailability; GI=gastrointestinal; HTE=heavily treatment experienced; IIV=interindividual variability; Kₐ=absorption rate constant; K_{dir}=direct absorption rate constant; Kₜᵣ=transit absorption rate constant; PK=pharmacokinetic; Q=intercompartmental CL; V_{c}=volume of central compartment; Vₚ=volume of peripheral compartment.

The effects of weight on CL, V_{c}, Vₚ, and Q were included using fixed allometric exponents of 0.75 and 1 for CL and volume of distribution, respectively; dose was found to affect Fₚₒ, and age, sex, and dose were found to affect CL.

TABLE 1

| Parameter | HTE | |
|---|---|---|
| θ₁: CL, L/h | 3.62 | (9.9%) |
| θ₂: V_{c}, L | 67.9 | (6%) |
| θ₃: PO Kₐ, 1/h | 0.0286 | (8.2%) |
| θ₄: Vₚ, L | 908 | (10.1%) |
| θ₅: Q, L/h | 41.2 | (5.3%) |
| θ₆: SC Kₜᵣ, 1/h | 0.00202 | (3.1%) |
| θ₇: SC K_{dir}, 1/h | 0.0038 | (7.9%) |
| θ₈: SC fraction for direct absorption | 0.421 | (5.6%) |
| θ₉: Fₚₒ relative to IV | 0.0627 | (9.1%) |
| θ₁₀: F_{sc} relative to IV | 1.02 | (8.1%) |
| θ₁₁: dose effect on Fₚₒ | −0.414 | (11.5%) |
| θ₁₂: dose effect on CL | 0.0887 | (33.7%) |
| θ₁₃: HV effect on CL | 0.462 | (26.6%) |
| θ₁₄: weight effect on CL and Q | 0.75 | |
| θ₁₅: weight effect on V_{c} and Vₚ | 1 | |
| θ₁₆: HV effect on Vₚ | 1.35 | (15.9%) |
| θ₁₇: age effect on CL | −0.165 | (64.8%) |
| θ₁₈: female effect on CL | −0.193 | (25.4%) |
| θ₁₉: pharmacoenhancer/booster effect on Fₚₒ | 0.534 | (35%) |
| θ₂₀: SC 150 mg/mL effect on Vₚ | −0.795 | (5.8%) |
| θ₂₁: SC 150 mg/mL effect on Kₜᵣ | 0.433 | (26.3%) |
| θ₂₂: CL effect on TN | 0.161 | (64.6%) |
| ω₁₁: IIV on CL, % CV | 43.2 | (4.6%) |
| ω₂₂: IIV on V_{c}, % CV | 0% | |
| ω₃₃: IIV on Vₚ, % CV | 85.4 | (5.5%) |
| ω₄₄: IIV on Kₜᵣ, % CV | 32.4 | (8.9%) |
| ω₅₅: IIV on Kₐ, % CV | 78.4 | (4.5%) |
| ω₆₆: IIV on K_{dir}, % CV | 0% | |
| ω₇₇: IIV on F_{sc}, % CV | 38.9% | (9.6%) |
| σ₁: residual proportion variability, % CV | 27.3% | (2%) |

| | |
|---|---|
| σ₂: residual additive, ng/mL | 0.05 |

Simulations were conducted to evaluate lenacapavir exposures for the dosing scenarios presented below.

### Scenario 1

Days 1 and 2: 600 mg oral, once-daily (QD)
Day 8: 300 mg oral, once-weekly (QW) (7th day from the first dose)

### Scenario 2

Days 1 and 2: 600 mg QD oral
Day 8: 300 mg QW oral (7th day from the first dose)

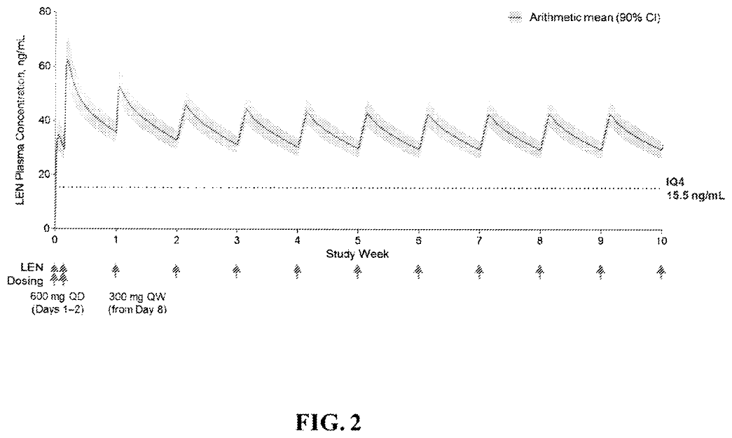

Simulations showed that an oral loading dose of 600 mg on Days 1 and 2 followed by oral 300-mg QW doses maintained the lower bound of the 90% CI of mean trough concentration above IQ4 (15.5 ng/mL) through the dosing interval; this regimen reached IQ4 rapidly within 4 hours (see FIG. 2).

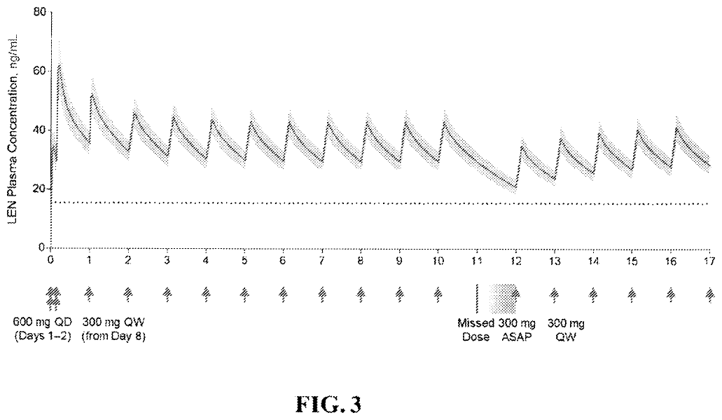

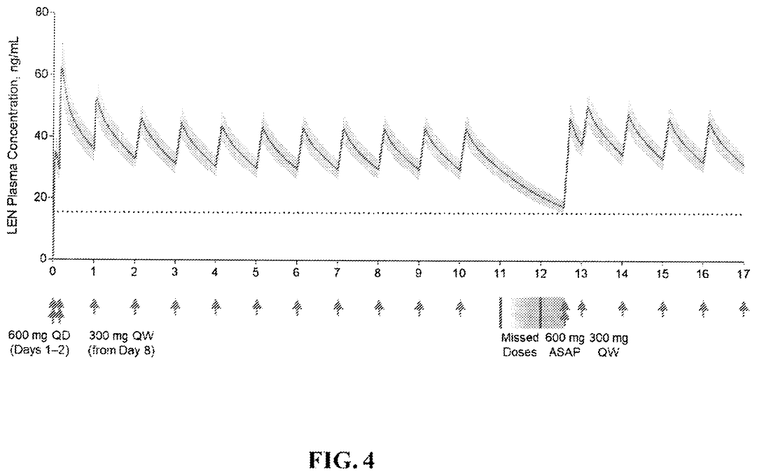

As shown in FIG. 3, simulations suggested that LEN 300 mg QW allows for a 7-day forgiveness window. If 1 oral LEN dose is missed, it should be taken as soon as possible and then normal dosing regimen can be resumed (i.e., taking 1 dose on the scheduled day) to maintain mean LEN concentrations above IQ4. If two oral LEN doses are missed, simulations indicated taking 2 doses as soon as possible and then resuming the normal regimen on the scheduled day will result in concentrations above IQ4 and within the safety margin (see FIG. 4). If taking doses on the scheduled dosing day, only 2 doses should be taken; never take 3 doses on the same day.

These data suggest that an oral LEN QW regimen (600 mg on Days 1 and 2, followed by 300 mg QW) is expected to rapidly achieve and then maintain LEN concentrations above IQ4 for the dosing interval and that the oral LEN 300 mg QW dosing allows for a 7-day forgiveness window for people with HIV after the last missed dose.

### Example 2. Spray-Dried Dispersion (SDD) Tablets

A tablet, prepared from a spray-dried disperstion of a sodium salt of the compound of Formula Ia, was prepared in a composition shown in Table 2 below (with an outer film coating made of Opadry® II White 85F18422, wherein the outer film coating provides a 3% weight gain based on the uncoated tablet).

TABLE 2
Composition of SDD tablets at 300 mg fixed dose

| Ingredient | Amount (w/w %) |
|---|---|
| Sodium salt of compound of Formula Ia | 20.46 |
| Copovidone | 4.88 |
| Poloxamer 407 | 1.33 |
| Microcrystalline Cellulose | 21.28 |
| Mannitol | 42.55 |
| Croscarmellose Sodium | 8.00 |
| Magnesium Stearate | 1.50 |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1-2. (canceled)

3. A method of preventing human immunodeficiency virus (HIV) infection in a patient, comprising administering to the patient a compound of Formula Ia: 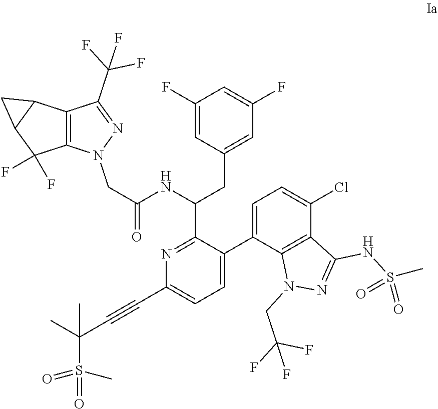 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein the method comprises pre-exposure prophylaxis (PrEP).

4. A method of preventing human immunodeficiency virus (HIV) infection in a patient, comprising administering to the patient a compound of Formula Ia: 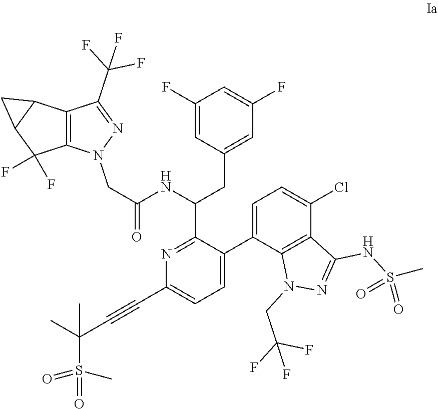 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt thereof, the method comprising:
(i) orally administering to the patient an initiation dosage comprising about 500 mg to about 700 mg of the compound of Formula Ia, or a pharmaceutically acceptable salt thereof, for a first period of time; and
(ii) orally administering to the patient one or more maintenance dosages each comprising about 200 mg to about 400 mg of the compound of Formula Ia, or a pharmaceutically acceptable salt thereof, for a second period of time, wherein the second period of time occurs after the first period of time;
wherein if the patient misses a maintenance dosage, the method further comprises orally administering to the patient about 200 mg to about 700 mg of the compound of Formula Ia, or a pharmaceutically acceptable salt thereof, for a third period of time, and resuming administration of the one or more maintenance dosages within about 1 day to about 7 days after the oral administration during the third period of time; and
wherein the method comprises pre-exposure prophylaxis (PrEP).

5. The method of claim 3, wherein the initiation dosage comprises orally administering to the patient about 600 mg of the compound of Formula Ia, or a pharmaceutically acceptable salt thereof, for the first period of time.

6. (canceled)

7. The method of claim 3, wherein the first period of time is about one day to about two days.

8. (canceled)

9. The method of claim 3, wherein the one or more maintenance dosages each comprise orally administering to the patient about 300 mg of the compound of Formula Ia, or a pharmaceutically acceptable salt thereof, for the second period of time.

10. (canceled)

11. The method of claim 3, wherein the second period of time begins about six days to about eight days after the first oral administration of the initiation dosage.

12. (canceled)

13. The method of claim 3, wherein the second period of time comprises continuous administration of the one or more maintenance dosages during the life of the patient.

14. The method of claim 4, wherein the third period of time begins within about one day to about fourteen days after the patient first misses a maintenance dosage.

15. The method of claim 4, wherein if the patient misses a maintenance dosage, the method further comprises orally administering to the patient about 300 mg to about 600 mg of the compound of Formula Ia, or a pharmaceutically acceptable salt thereof, for a third period of time, and resuming administration of the one or more maintenance dosages within about 1 day to about 7 days after the oral administration during the third period of time.

16-19. (canceled)

20. The method of claim 4, wherein the third period of time is about one day to about two days.

21-24. (canceled)

25. The method of claim 3, wherein the method comprises:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein the method comprises pre-exposure prophylaxis (PrEP).

26. The method of claim 4, wherein the method comprises:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses a maintenance dosage, the method further comprises orally administering to the patient about 300 mg of the compound of Formula Ia, or a pharmaceutically acceptable salt thereof, within a third period of time which is about one day to about fourteen days after the patient first misses the maintenance dosage, and resuming administration of the one or more maintenance dosages within about 1 day to about 7 days after the oral administration during the third period of time; and
wherein the method comprises pre-exposure prophylaxis (PrEP).

27. The method of claim 4, wherein the method comprises:
(i) orally administering to the patient an initiation dosage comprising about 600 mg of the compound of Formula Ia, or a pharmaceutically acceptable salt thereof, for two days; and
(ii) orally administering to the patient one or more maintenance dosages each comprising about 300 mg of the compound of Formula Ia, or a pharmaceutically acceptable salt thereof, once per week;
wherein if the patient misses two consecutive maintenance dosages, the method further comprises orally administering to the patient about 600 mg of the compound of Formula Ia, or a pharmaceutically acceptable salt thereof, within a third period of time which is about one day to about fourteen days after the patient first misses the second maintenance dosage, and resuming administration of the one or more maintenance dosages within about 1 day to about 7 days after the oral administrations during the third period of time; and
wherein the method comprises pre-exposure prophylaxis (PrEP).

28. The method of claim 3, wherein the compound of Formula Ia is administered as the sodium salt.

29. The method of claim 3, wherein each oral administration is administered as a tablet comprising the sodium salt of the compound of Formula Ia.

30. The method of claim 29, wherein each tablet is prepared from a spray-dried dispersion technology.

31. The method of claim 29, wherein each tablet comprises about 5 w/w % to about 45 w/w % of the sodium salt of the compound of Formula Ia, about 1 w/w % to about 10 w/w % of copovidone, about 0.01 w/w % to about 10 w/w % of poloxamer 407, about 5 w/w % to about 45 w/w % of microcrystalline cellulose, about 15 w/w % to about 70 w/w % of mannitol, about 1 w/w % to about 30 w/w % of croscarmellose sodium, and about 0.01 w/w % to about 10 w/w % of magnesium stearate, and one or more pharmaceutically acceptable excipients.

32. (canceled)

33. The method of claim 29, wherein each tablet comprises about 300 mg of the compound of Formula Ia, or a pharmaceutically acceptable salt thereof.

34. The method of claim 29, wherein each tablet comprises about 306.8 mg of the sodium salt of the compound of Formula Ia.

35. The method of claim 29, wherein each tablet further comprises an outer film coat.

36. The method of claim 35, wherein the outer film coat provides from about 1% to about 8% weight gain based on the uncoated tablet.

37. (canceled)

38. The method of claim 3, wherein
the compound of Formula Ia, or a pharmaceutically acceptable salt thereof, is administered as a monotherapy.

39. The method of claim 3, wherein the method comprises event driven administration of the compound of Formula Ia, or a pharmaceutically acceptable salt thereof, to the patient.

40. (canceled)

41. The method of claim 3, wherein the method further comprises post-exposure prophylaxis (PEP).

42-46. (canceled)

47. The method of claim 3, wherein the pre-exposure prophylaxis (PrEP) comprises continuous PrEP.

48-84. (canceled)

85. The method of claim 3, wherein the compound of Formula Ia, or a pharmaceutically acceptable salt thereof, is a compound of Formula Ib: 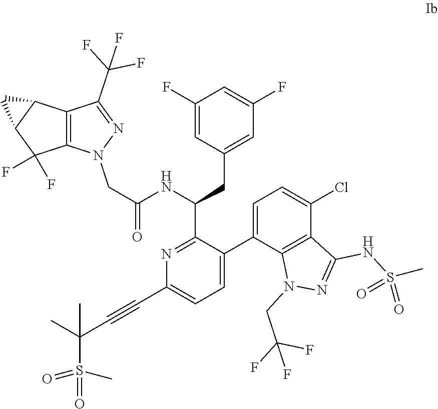 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]
or a pharmaceutically acceptable salt thereof.

86. The method of claim 85, wherein the compound of Formula Ib is administered as the sodium salt.

87. The method of claim 29, wherein each tablet comprises about 15 w/w % to about 25 w/w % of the sodium salt of the compound of Formula Ia, about 3 w/w % to about 6 w/w % copovidone, about 0.5 w/w % to about 3.0 w/w % poloxamer 407, about 18 w/w % to about 30 w/w % microcrystalline cellulose, about 40 w/w % to about 50 w/w % mannitol, about 6 w/w % to about 10 w/w % croscarmellose sodium, and about 1.0 w/w % to about 3.0 w/w % magnesium stearate.

88. The method of claim 29, wherein each tablet comprises about 20.46 w/w % of the sodium salt of the compound of Formula Ia, about 4.88 w/w % copovidone, about 1.33 w/w % poloxamer 407, about 21.28 w/w % microcrystalline cellulose, about 42.55 w/w % mannitol, about 8.00 w/w % croscarmellose sodium, and about 1.50 w/w % magnesium stearate.

89. The method of claim 88, wherein each tablet comprises about 306.8 mg of the sodium salt of the compound of Formula Ia.

90. The method of claim 88, wherein each tablet further comprises an outer film coat.

91. The method of claim 90, wherein the outer film coat provides from about 1% to about 8% weight gain based on the uncoated tablet.

92. The method of claim 90, wherein the outer film coat provides about 4% weight gain based on the uncoated tablet.
